(19) United States
(12) Patent Application Publication
(10) Pub. No.: US 2019/0290182 A1
(43) Pub. Date: Sep. 26, 2019

(54) METHOD OF DIAGNOSTIC RELEVANCE BASED ON TASTE RECOGNITION

(71) Applicant: RANVIER HEALTH LIMITED, Bristol (GB)

(72) Inventors: Jan MELICHAR, Bristol (GB); Lucy DONALDSON, Bristol (GB); David ADAMS, Bristol (GB); Michael HARTNACK, Bristol (GB)

(73) Assignee: RANVIER HEALTH LIMITED, Bristol (GB)

(21) Appl. No.: 16/316,739

(22) PCT Filed: Jul. 11, 2017
(86) PCT No.: PCT/GB2017/052034

§ 371 Date: Jan. 10, 2019

(30) Foreign Application Priority Data
Jul. 11, 2016 (EP) 16178920.1

(51) Int. Cl. A61B 5/00; A61B 5/16

(52) U.S. Cl. CPC A61B 5/4017; A61B 5/16

(57) ABSTRACT

A method for determining neurotransmitter levels and/or neurotransmitter receptor sensitivity in an individual, comprising: determining a taste recognition profile of the individual for at least one taste modality prior and subsequent to the administration of a pharmaceutical for increasing neurotransmission; comparing the taste recognition profiles to determine a change; and comparing that change with corresponding measurements from a comparative database to determine neurotransmitter levels and/or neurotransmitter receptor sensitivity in the individual. In addition, a sample of a taste modality is provided for use in a method of diagnosis of a psychiatric, neurological, psychosomatic or physical disorder in an individual.

### FIELD OF THE INVENTION

The present invention relates to a method of determining neurotransmitter levels and/or neurotransmitter receptor sensitivity in an individual. The invention also relates to a method of determining a change in neurotransmitter levels and/or neurotransmitter receptor sensitivity in an individual. The invention further relates to a method for determining whether an individual suffering from a psychiatric, neurological, psychosomatic or physical disorder is not suitable for treatment with a pharmaceutical for increasing neurotransmission. In addition, the invention relates to a sample of a taste modality for use in a method of diagnosis of a psychiatric, neurological, psychosomatic or physical disorder in an individual.

### BACKGROUND OF THE INVENTION

Taste perception in humans is known to be plastic and can change over time. In particular, taste perception (including taste thresholds, perceived intensity and pleasantness or unpleasantness of taste) can be altered by different states in both health and disease.

Monoamine neurotransmitters, which include noradrenaline (NA) and serotonin (5-HT), are released from neurones in the brain and contribute to mental state, well-being and perception of the human senses. These same monoamine neurotransmitters are used by the taste bud on the tongue in the appreciation of taste. Manipulation of the monoamine neurotransmitters, whether by normal physiological cycles (e.g. the menstrual cycle), disease state (e.g. depression) or by pharmacological challenge (e.g. by treatment with antidepressants that modulate monoamine neurotransmission) can lead to changes in mental state and well-being.

The “monoamine theory of depression” suggests that depression is a consequence of diminished circulating monoamine concentrations and hence neurotransmission of NA, dopamine and 5-HT, and/or a reduction in the sensitivity of their receptors (Hirschfeld, 2000. J Clin Psychiatry 61 [Suppl 6]:4-6). Antidepressants used in the treatment of major depression disorder (MDD) target the monoamine neurotransmitters (i.e. NA and 5-HT).

Heath et al., 2006. J. Neurosci 6; 26(49):12664-71. investigated plasticity in the human taste system of healthy volunteers by modulating systemic monoamines using acute dosages of specific monoamine reuptake inhibitors. Psychophysical taste functions were determined before, and two hours after, administration of a 5-HT-specific reuptake inhibitor (SSRI; paroxetine), an NA reuptake inhibitor (NARI; reboxetine) or an inactive placebo (lactose). It was found that enhancement of 5-HT significantly reduced bitter and sweet taste thresholds and enhancement of NA significantly reduced bitter and sour thresholds in healthy individuals. Furthermore, non-clinical anxiety levels (although within the normal range) of the subjects were positively correlated with bitter and salt taste thresholds. In other words, individuals with higher non-clinical anxiety levels had higher taste thresholds. Although the results of this study indicate that human taste thresholds are plastic and can be lowered by modulation of monoamines, no conclusions could be drawn as to whether there was a relationship between clinical depression and taste in the subjects because all subjects had very low scores on the Beck's Depression Inventory questionnaire.

Yoshitake et al. 2006, Biomed Chromatogr. March; 20(3):267-81 relates to determination of monoamine neurotransmitters in microdialysis samples from rat and mouse brains, Microdialysis allows sampling of molecules transported into, or generated with, the extracellular space of the brain. There remains a need for non-invasive methods of determining the level of monoamine neurotransmitters in a patient.

Melichar J. K. et al. 2011 Journal of Psychopharmacology vol. 25, no. 3, Suppl. S, page A21 is an abstract that sets out preliminary findings. It is noted that compared with healthy controls, depressed patients had blunted sucrose and salt thresholds. There remains a need for a robust and objective assessment of depression.

The diagnosis of clinical depression is currently problematic, with high proportions of patients being prescribed antidepressants for which they have no medical need, and of depressed patients who go untreated. Currently patients suspected of suffering from clinical depression are prescribed an antidepressant by their doctor and their symptoms are monitored (often on an ad hoc basis) over a period of weeks and months. However, there are different classes of antidepressant that elevate either 5-HT or NA or both, in different amounts. Doctors are unable to ascertain whether a patient presenting with depressive symptoms is biologically depressed (i.e. depleted in either 5-HT and/or NA or having reduced sensitivity to these monoamines). Therefore, the doctor must make a choice of antidepressant or psychological treatments based on clinical assessment of symptoms (i.e. whether or not they believe that the patient is biologically depressed) and then monitor the response to treatment. This results in up to half of patients not responding to initial treatment.

A lack of response does not necessarily mean the patient is not depressed. It may alternatively mean that they may have been given an incorrect antidepressant treatment. Furthermore, due to the way antidepressants work, a patient is likely to experience negative side effects from the antidepressant (for example, anxiety, insomnia, loss of libido, gastro-intestinal problems) immediately with any benefits following days to weeks later. This often results in patients discontinuing treatment before any positive results have been experienced.

Clinical depression is a disease where early diagnosis and appropriate treatment has been shown to improve patient outcomes significantly. An objective assessment of monoamine levels and/or monoamine receptor sensitivity in an individual (i.e. an indicator of whether or not a patient is biologically depressed) would facilitate the early diagnosis of clinical depression and the selection of an appropriate treatment. The present invention seeks to provide a solution to this problem.

The present invention arises out of the surprising finding that individuals with previously undiagnosed and untreated clinical depression exhibit an acute response to pharmacological challenge (i.e. with a pharmaceutical that increases monoamine neurotransmission) when taste recognition profiles are measured, despite a lack of immediate effect on their symptoms. This finding led to the surprising realisation that determining the taste recognition profile of individuals could be used to determine monoamine levels and/or monoamine receptor sensitivity objectively in an individual and hence could be used as an indicator of clinical depression and/or a clinical anxiety disorder in an individual.

### SUMMARY OF THE INVENTION

In accordance with a first aspect of the invention, there is provided a method for determining neurotransmitter levels and/or neurotransmitter receptor sensitivity in an individual, the method comprising the steps of:

(a) determining a taste recognition profile of the individual for at least one taste modality prior to an administration of a pharmaceutical for increasing neurotransmission;
(b) determining a taste recognition profile of the individual for the at least one taste modality subsequent to the administration of the pharmaceutical for increasing neurotransmission;
(c) performing a comparison between the taste recognition profile of step (a) and the taste recognition profile of step (b) to determine a change in taste recognition profile of the individual to the at least one taste modality;
(d) performing a comparison between the change in taste recognition profile and corresponding measurements from a comparative database to determine neurotransmitter levels and/or neurotransmitter receptor sensitivity in the individual.

Preferably, the at least one taste modality comprises a sweet taste modality and the comparison between the change in taste recognition profile and corresponding measurements from the comparative database indicates an increased taste sensitivity for the sweet taste modality,

wherein the increased taste sensitivity is an indication of reduced neurotransmitter levels and/or neurotransmitter receptor sensitivity in the individual prior to the administration of the pharmaceutical for increasing neurotransmission.

Advantageously, the taste recognition profile is a taste recognition threshold.

Conveniently, the neurotransmitter is a monoamine and the pharmaceutical for increasing neurotransmission is a pharmaceutical for increasing monoamine neurotransmission.

Preferably, the individual is suffering from a psychiatric, neurological, psychosomatic or physical disorder.

Conveniently, the increased taste sensitivity for the sweet taste modality is an indication that the individual is suffering from a psychiatric, neurological, psychosomatic or physical disorder that is suitable for treatment with a pharmaceutical for increasing monoamine neurotransmission.

Alternatively, the comparison between the change in taste recognition threshold and corresponding measurements from the comparative database indicates a less than 10% change or a decreased taste sensitivity for the at least one taste modality, and wherein the less than 10% change or the decreased taste sensitivity to the at least one taste modality is an indication that the individual is suffering from a psychiatric, neurological, psychosomatic or physical disorder that is not suitable for treatment with a pharmaceutical for increasing monoamine neurotransmission.

Preferably, the at least one taste modality comprises a sweet taste modality.

In accordance with a second aspect of the invention, there is provided a method for determining a change in neurotransmitter levels and/or neurotransmitter receptor sensitivity in an individual, the method comprising the steps of:

(a) at a first point in time, determining a first taste recognition profile of the individual for at least one taste modality subsequent to an administration of a pharmaceutical for increasing neurotransmission;
(b) at a second point in time, determining a second taste recognition profile of the individual for the at least one taste modality subsequent to an administration of the pharmaceutical for increasing neurotransmission;
(c) comparing the first taste recognition profile with the second taste recognition profile to determine the change in neurotransmitter levels and/or neurotransmitter receptor sensitivity in the individual.

Advantageously, the method further comprises, prior to step (a), the step of:

determining a taste recognition profile of the individual for at least one taste modality prior to the administration of the pharmaceutical for increasing neurotransmission; and, performing a comparison between said taste recognition profile and the first taste recognition profile and/or the second taste recognition profile to determine further changes in neurotransmitter levels and/or neurotransmitter receptor sensitivity in the individual.

Conveniently, the method in accordance with the first or second aspect of the invention further comprises administering the pharmaceutical for increasing neurotransmission.

In accordance with a third aspect of the invention, there is provided a method for determining whether an individual suffering from a psychiatric, neurological, psychosomatic or physical disorder is not suitable for treatment with a pharmaceutical for increasing neurotransmission, the method comprising the steps of:

(a) determining a taste recognition profile of the individual for at least one taste modality prior to an administration of the pharmaceutical for increasing neurotransmission;
(b) determining a taste recognition profile of the individual for the at least one taste modality subsequent to the administration of the pharmaceutical for increasing neurotransmission;
(c) performing a comparison between the taste recognition profile of step (a) and the taste recognition profile of step (b) to determine a change in taste recognition profile of the individual to the at least one taste modality;
(d) performing a comparison between the change in taste recognition profile and corresponding measurements from a comparative database to determine whether the individual is suffering from a psychiatric, neurological, psychosomatic or physical disorder that is not suitable for treatment with the pharmaceutical for increasing neurotransmission.

Preferably, the at least one taste modality comprises a sweet taste modality and the comparison between the change in taste recognition profile and corresponding measurements from the comparative database indicates a less than 10% change or a decreased taste sensitivity for the sweet taste modality,

wherein the less than 10% change or the decreased taste sensitivity to the sweet taste modality is an indication that the individual is suffering from a psychiatric, neurological, psychosomatic or physical disorder that is not suitable for treatment with the pharmaceutical for increasing neurotransmission.

In accordance with a fourth aspect of the invention, there is provided a method for determining whether an individual suffering from a psychiatric, neurological, psychosomatic or physical disorder is not suitable for treatment with a pharmaceutical for increasing neurotransmission, the method comprising the steps of:

(a) determining a taste recognition profile of the individual for first and second taste modalities prior to an administration of the pharmaceutical for increasing neurotransmission;
(b) determining a taste recognition profile of the individual for first and second taste modalities subsequent to the administration of the pharmaceutical for increasing neurotransmission;
(c) performing a comparison between the taste recognition profiles of step (a) and the taste recognition profiles of step (b) to determine a first change in taste recognition profile of the individual to the first taste modality and a second change in taste recognition profile of the individual to the second taste modality;
(d) performing a comparison between the first change in taste recognition profile and the second change in taste recognition profile to determine whether the individual is suffering from a psychiatric, neurological, psychosomatic or physical disorder that is not suitable for treatment with the pharmaceutical for increasing neurotransmission.

Advantageously, the first and second taste modalities are sweet and salt respectively and the comparison of step (d) indicates a relatively small or negative first change in taste recognition profile and a relatively large second change in taste recognition profile,

wherein the relatively small or negative first change in taste recognition profile and the relatively large second change in taste recognition profile is an indication that the individual is suffering from a psychiatric, neurological, psychosomatic or physical disorder that is not suitable for treatment with the pharmaceutical for increasing neurotransmission.

Conveniently, the relatively small first change is a less than 10% change in taste recognition profile; and the relatively large second change is a more than 10% change in taste recognition profile.

Alternatively, the second taste modality is bitter or sour.

In accordance with a fifth aspect of the invention, there is provided a sample of a taste modality for use in a method of diagnosis of a psychiatric, neurological, psychosomatic or physical disorder in an individual, wherein the method comprises the steps of:

(a) contacting the taste modality with the individual's tongue and determining a taste recognition profile of the individual for the taste modality prior to an administration of a pharmaceutical for increasing neurotransmission;
(b) contacting the taste modality with the individual's tongue and determining a taste recognition profile of the individual for the taste modality subsequent to the administration of the pharmaceutical for increasing neurotransmission;
(c) performing a comparison between the taste recognition profile of step (a) and the taste recognition profile of step (b) to determine a change in taste recognition profile of the individual to the taste modality;
(d) performing a comparison between the change in taste recognition profile and corresponding measurements from a comparative database to diagnose a psychiatric, neurological, psychosomatic or physical disorder in the individual.

Advantageously, the taste modality is a sweet taste modality and the comparison between the change in taste recognition profile and corresponding measurements from the comparative database indicates an increased taste sensitivity to the sweet taste modality

wherein the increased taste sensitivity is an indication of a psychiatric, neurological, psychosomatic or physical disorder in the individual.

Preferably, the sample of the taste modality is for use in a method of in vivo diagnosis of a disorder in which monoamine levels and/or monoamine receptor sensitivity is deregulated in an individual, wherein the method comprises the steps of:

(a) contacting the taste modality with the individual's tongue and determining a taste recognition threshold of the individual for the taste modality prior to an administration of a pharmaceutical for increasing monoamine neurotransmission;
(b) contacting the taste modality with the individual's tongue and determining a taste recognition threshold of the individual for the taste modality subsequent to the administration of the pharmaceutical for increasing monoamine neurotransmission;
(c) performing a comparison between the taste recognition threshold of step (a) and the taste recognition threshold of step (b) to determine a change in taste recognition threshold of the individual to the taste modality;
(d) performing a comparison between the change in taste recognition threshold and corresponding measurements from a comparative database to diagnose a disorder in which monoamine levels and/or monoamine receptor sensitivity is deregulated in the individual.

Advantageously, the taste modality is a sweet taste modality and the comparison between the change in taste recognition threshold and corresponding measurements from the comparative database indicates an increased taste sensitivity to the sweet taste modality

wherein the increased taste sensitivity is an indication of a disorder in which monoamine levels and/or monoamine receptor sensitivity is deregulated in the individual.

Conveniently, the disorder in which monoamine levels and/or monoamine receptor sensitivity is deregulated is a psychiatric, neurological, psychosomatic or physical disorder.

In accordance with a sixth aspect of the invention, there is provided a method for determining whether an individual suffering from a psychiatric, neurological, psychosomatic or physical disorder is suitable for treatment with a pharmaceutical for increasing neurotransmission, the method comprising the steps of:

(a) determining a taste recognition profile of the individual for at least one taste modality prior to an administration of the pharmaceutical for increasing neurotransmission;
(b) determining a taste recognition profile of the individual for the at least one taste modality subsequent to the administration of the pharmaceutical for increasing neurotransmission;
(c) performing a comparison between the taste recognition profile of step (a) and the taste recognition profile of step (b) to determine a change in taste recognition profile of the individual to the at least one taste modality;
(d) performing a comparison between the change in taste recognition profile and corresponding measurements from a comparative database to determine whether the individual is suffering from a psychiatric, neurological, psychosomatic or physical disorder that is suitable for treatment with the pharmaceutical for increasing neurotransmission.

Preferably, the at least one taste modality comprises a sweet taste modality and the comparison between the change in taste recognition profile and corresponding measurements from the comparative database indicates a more than 10% change or a increased taste sensitivity for the sweet taste modality,

wherein the more than 10% change or the increased taste sensitivity to the sweet taste modality is an indication that the individual is suffering from a psychiatric, neurological, psychosomatic or physical disorder that is suitable for treatment with the pharmaceutical for increasing neurotransmission.

In accordance with a seventh aspect of the invention, there is provided a method for determining whether an individual suffering from a psychiatric, neurological, psychosomatic or physical disorder is suitable for treatment with a pharmaceutical for increasing neurotransmission, the method comprising the steps of:

(a) determining a taste recognition profile of the individual for first and second taste modalities prior to an administration of the pharmaceutical for increasing neurotransmission;
(b) determining a taste recognition profile of the individual for first and second taste modalities subsequent to the administration of the pharmaceutical for increasing neurotransmission;
(c) performing a comparison between the taste recognition profiles of step (a) and the taste recognition profiles of step (b) to determine a first change in taste recognition profile of the individual to the first taste modality and a second change in taste recognition profile of the individual to the second taste modality;
(d) performing a comparison between the first change in taste recognition profile and the second change in taste recognition profile to determine whether the individual is suffering from a psychiatric, neurological, psychosomatic or physical disorder that is suitable for treatment with the pharmaceutical for increasing neurotransmission.

Advantageously, the first and second taste modalities are sweet and salt respectively and the comparison of step (d) indicates a relatively large or positive first change in taste recognition profile and a relatively small second change in taste recognition profile,

wherein the relatively large or positive first change in taste recognition profile and the relatively small second change in taste recognition profile is an indication that the individual is suffering from a psychiatric, neurological, psychosomatic or physical disorder that is suitable for treatment with the pharmaceutical for increasing neurotransmission.

Conveniently, the relatively large first change is a more than 10% change in taste recognition profile; and the relatively small second change is a less than 10% change in taste recognition profile.

Alternatively, the second taste modality is bitter or sour.

Advantageously, the method in accordance with the sixth or seventh aspect of the invention further comprises administering the pharmaceutical for increasing neurotransmission.

Conveniently, the method in accordance with the sixth or seventh aspect of the invention further comprises: (e) administering an effective amount of a pharmaceutical for increasing neurotransmission to the suitable individual.

Preferably, the psychiatric disorder is clinical depression or a clinical anxiety disorder.

Conveniently, the comparison is performed using a means for comparison.

Preferably, the taste recognition profile is a taste recognition threshold.

Advantageously, the neurotransmitter is a monoamine and the pharmaceutical for increasing neurotransmission is a pharmaceutical for increasing monoamine neurotransmission.

In accordance with an eight aspect of the invention, there is provided a method for determining monoamine levels and/or monoamine receptor sensitivity in an individual suffering from a psychiatric, neurological, psychosomatic or physical disorder, the method comprising the steps of:

(a) determining a taste recognition threshold of the individual for at least one taste modality prior to an administration of a pharmaceutical for increasing monoamine neurotransmission;
(b) determining a taste recognition threshold of the individual for the at least one taste modality subsequent to the administration of the pharmaceutical for increasing monoamine neurotransmission;
(c) performing a comparison between the taste recognition threshold of step (a) and the taste recognition threshold of step (b) to determine a change in taste recognition threshold of the individual to the at least one taste modality,
wherein the change in the taste recognition threshold is used to determine monoamine levels and/or monoamine receptor sensitivity in the individual.

Preferably, the change in taste recognition threshold is an increased taste sensitivity to the at least one taste modality and the increased taste sensitivity is an indication of reduced monoamine levels and/or monoamine receptor sensitivity in the individual prior to the administration of the pharmaceutical for increasing monoamine neurotransmission.

Conveniently, the increased taste sensitivity is an indication that the individual is suffering from a psychiatric, neurological, psychosomatic or physical disorder that is suitable for treatment with a pharmaceutical for increasing monoamine neurotransmission.

Alternatively, the change in taste recognition threshold is a less than 10% change or a decreased taste sensitivity to the at least one taste modality, and

wherein the less than 10% change or the decreased taste sensitivity to the at least one taste modality is an indication that the individual is suffering from a psychiatric, neurological, psychosomatic or physical disorder that is not suitable for treatment with a pharmaceutical for increasing monoamine neurotransmission.

Advantageously, the at least one taste modality comprises a sweet taste modality.

Preferably, the psychiatric disorder is clinical depression or a clinical anxiety disorder.

Conveniently, the comparison is performed using a means for comparison.

Conveniently, the pharmaceutical for increasing monoamine neurotransmission is a dopamine, noradrenaline or serotonin reuptake inhibitor.

Preferably, there is a time lag of approximately two hours between steps (a) and (b) of the method of the first, third, fourth, fifth, sixth and seventh aspects of the invention.

Preferably, there is also time lag of approximately two hours between steps (a) and (b) of the method of the eight aspect of the invention.

The term “neurotransmitter” as used herein refers, in some embodiments, to a substance that is released on excitation from the axon terminal of a presynaptic neuron of the central or peripheral nervous system and travels across the synaptic cleft either to elicit or inhibit its target cell.

The term “neurotransmitter levels” as used herein refers, in some embodiments, to the concentration of a neurotransmitter in the blood plasma or in the central or peripheral nervous system of an individual.

The term “neurotransmitter receptor sensitivity” as used herein refers, in some embodiments, to the ability of a protein receptor on the membrane of a presynaptic or postsynaptic cell to bind selectively to a neurotransmitter (i.e. its ligand) and/or to produce a specific physiological effect that accompanies the binding. A reduction in neurotransmitter receptor sensitivity results in a corresponding reduction in the specific physiological effect that accompanies the binding. An increase in neurotransmitter receptor sensitivity results in a corresponding increase in the specific physiological effect that accompanies the binding. In one embodiment, the term “neurotransmitter receptor sensitivity” encompasses the specificity of neurotransmitter receptor for its neurotransmitter ligand. In another embodiment, the term “neurotransmitter receptor sensitivity” encompasses the affinity of a neurotransmitter receptor for its neurotransmitter ligand.

The term “a taste recognition profile” as used herein refers, in some embodiments, to characteristics of an individual's response to a taste modality. In one embodiment, an individual's sensitivity to a taste modality is determined by testing at least three concentrations of a taste modality on the tip of the tongue of the individual. The at least three concentrations may be provided in a mixed sequence that includes other intervening taste modalities. The percentage of positive recognition/identification of the concentration of the taste modality is plotted on a graph against log solute concentration of the taste modality. A curve is then fitted to the data which represents the individual's taste recognition profile to that taste modality. The taste recognition profile encompasses an individual's “taste recognition threshold” which is calculated from the aforementioned curve and, as used herein, refers to the concentration at which an individual would correctly identify a taste 50% of the time.

The term “taste” as used herein refers to the sense effected by the gustatory receptors on the tongue. Furthermore, the term “taste modality” as used herein refers, in some embodiments, to one of the five qualities which can be distinguished by the sense of taste, namely, sweet, sour, salt, bitter and umami.

The term “increased taste sensitivity” as used herein refers, in some embodiments, to a decrease in a second taste recognition threshold relative to a first taste recognition threshold. In other words, the change in taste recognition profile is positive. In one embodiment, the percentage change between the second taste recognition threshold relative to the first taste recognition threshold is at least a 10%, 25% or 50% decrease. In some other embodiments, a change in taste recognition profile, which is relatively large when compared with corresponding measurements from a comparative database is an indication of increased taste sensitivity. In one embodiment, the change in taste recognition profile is compared with a threshold value to determine an increased taste sensitivity. In one embodiment, the change in taste recognition profile is a change in taste recognition threshold (also referred to as “deltaTRT”). In one embodiment, the positive change in taste recognition threshold is statistically significant compared with the threshold value in order to correspond to “increased taste sensitivity”. In one embodiment, the threshold value is contained within a comparative database.

The term “a less than 10% change or a decreased taste sensitivity” as used herein refers, in some embodiments, to either a less than 10% change in a second taste recognition threshold compared with a first taste recognition threshold or to an increase in a second taste recognition threshold relative to a first taste recognition threshold, respectively. In some other embodiments, a change in taste recognition profile between a first and a second taste recognition profile is less than 10% in relation to the first taste recognition profile. In one embodiment, the change in taste recognition profile is a change in taste recognition threshold (also referred to as “deltaTRT”).

The term “a pharmaceutical for increasing neurotransmission” as used herein refers, in some embodiments, to a substance that enhances the activity of a neurotransmitter and hence promotes neurotransmission in an individual either by increasing release of the neurotransmitter into the synapse, blocking reuptake of the neurotransmitter, inhibiting metabolism of the neurotransmitter or acting directly on a receptor of the neurotransmitter. In one embodiment, the pharmaceutical for increasing neurotransmission is a monoamine reuptake inhibitor.

The term “a comparative database” as used herein refers, in some embodiments, to an organised collection of data, comprising data corresponding to the data of interest and allowing a comparative analysis to be performed. The comparative database may be refined over time. In one embodiment, the comparative database includes data from healthy individuals and/or from individuals suffering from a psychiatric, neurological, psychosomatic or physical disorder. In one embodiment, the comparative database comprises the change in taste recognition profile values determined in individuals using the methods of the present invention. In one embodiment, the change in taste recognition profile is a change in taste recognition threshold (also referred to as “deltaTRT”).

In an alternative embodiment, the term “a comparative database” refers to a collection of data from a single individual. In other words, the comparative database consists of data from an individual. In some embodiments, the comparative database comprises data on the change in taste recognition profiles of the individual to two or more taste modalities. In one embodiment, the change in taste recognition profile is a change in taste recognition threshold (also referred to as “deltaTRT”).

In a further embodiment, the comparative database consists of a single threshold value against which the change in taste recognition profile is compared. In one embodiment, the change in taste recognition profile is a change in taste recognition threshold (also referred to as “deltaTRT”).

The term “means for comparison” as used herein refers, in some embodiments, to a means having technical character for comparing data. In one embodiment, the means for comparison is a processor being configured to compare the data.

The phrase “a psychiatric, neurological, psychosomatic or physical disorder” as used herein refers, in some embodiments, to one of the following disorders. A psychiatric disorder refers to any pattern of psychological or behavioural symptoms that causes an individual significant distress, impairs their ability to function in life, and/or significantly increases their risk of death, pain, disability, or loss of freedom. Psychiatric disorders include clinical anxiety disorders and clinical depression. A neurological disorder refers to a disease of the central and peripheral nervous system. In other words, the brain, spinal cord, cranial nerves, peripheral nerves, nerve roots, autonomic nervous system, neuromuscular junction, and muscles. A psychosomatic disorder refers to a disorder in which an individual experiences physical symptoms which do not have a clear physical basis and which are, or which are believed to be, of mental origin. In one embodiment, the psychosomatic disorder is referred to by the individual as causing “total body pain” or the like (for example, at detailed in Fuller & Toon, 1988 “Medical Practice in a Multicultural Society”). A “physical disorder” as used herein refers to a disorder linked to altered neurotransmitter levels and/or neurotransmitter receptor sensitivity in which an individual experiences physical symptoms. Without wishing to be bound by theory, it is thought that a common mechanism underlying psychiatric, neurological, psychosomatic or physical disorders in certain individuals is the deregulation of monoamine neurotransmitter levels and/or monoamine neurotransmitter receptor sensitivity in the individual. The methods of the present invention are a useful indication as to whether or not an individual suffering from a psychiatric, neurological, psychosomatic or physical disorder exhibits deregulation of monoamine neurotransmitter levels and/or monoamine neurotransmitter receptor sensitivity and hence whether or not the individual is suitable for treatment with a pharmaceutical for increasing monoamine neurotransmission. The present invention is thought to be useful for individuals suffering from any disorder in which monoamine neurotransmitter levels and/or monoamine neurotransmitter receptor sensitivity is believed to be deregulated.

The phrase “not suitable for treatment with a pharmaceutical for increasing neurotransmission” as used herein refers, in some embodiments, to an individual suffering from symptoms suggesting a psychiatric, neurological, psychosomatic or physical disorder who would not benefit from treatment with a pharmaceutical for increasing neurotransmission. In other words, there is no underlying biological cause that could be improved by treatment with a pharmaceutical for increasing neurotransmission. In one embodiment, the individual is suffering from symptoms suggesting clinical depression as a result of social and/or external factors. The individual would not be suitable for treatment with a pharmaceutical for increasing neurotransmission as there is not, or there is not expected to be, an underlying biological cause of the symptoms that could be improved by the treatment.

The term “clinical depression” as used herein refers to a major depressive episode, as defined in DSM-5 (296) and ICD-10 (F32.2). Clinical depression is also known as major depressive disorder (MDD). Further details on the definition of clinical depression are provided in Cleare et al. J Psychopharmacol. 2015 May; 29(5):459-525, which is incorporated herein by reference.

The term “clinical anxiety disorder” as used herein refers to a clinically recognised biologically treatable anxiety disorder such as panic disorder (ICD-10 F41.0), generalised anxiety disorder (ICD-10 F41.1), obsessive compulsive disorder (ICD-10 F42) or social anxiety disorder (ICD-10 F40.1). Further details on the definitions of clinical anxiety disorders are provided in Baldwin et al. J Psychopharmacol. 2014 May; 28(5):403-39, which is incorporated herein by reference.

The term “monoamine” as used herein refers to a compound having a single amine group, especially one which is a neurotransmitter. Examples of monoamines include dopamine, noradrenaline (otherwise known as norepinephrine) or serotonin.

The term “a pharmaceutical for increasing monoamine neurotransmission” as used herein refers to a substance that enhances the activity of a monoamine and hence promotes monoamine neurotransmission in an individual either by increasing release of the monoamine into the synapse, blocking reuptake of the monoamine, inhibiting metabolism of the monoamine or acting directly on a receptor of the monoamine. In one embodiment, the pharmaceutical for increasing monoamine neurotransmission is a monoamine reuptake inhibitor.

The term “a monoamine reuptake inhibitor” as used herein refers to a substance that acts as a reuptake inhibitor of one or more of the three major monoamine neurotransmitters, serotonin, noradrenaline or dopamine by blocking the action of one or more of the respective monoamine transporters, which include the serotonin transporter, noradrenaline transporter, and dopamine transporter. This in turn results in an increase in the synaptic concentrations of one or more of these monoamine neurotransmitters and therefore an increase in neurotransmission. In one embodiment, the monoamine reuptake inhibitor is a serotonin-specific reuptake inhibitor (SSRI; for example, paroxetine). In an alternative embodiment, the monoamine reuptake inhibitor is a noradrenaline reuptake inhibitor (NARI; for example, reboxetine).

### BRIEF DESCRIPTION OF THE FIGURES

FIG. 1 is a perspective view of a device for determining a taste recognition profile of an individual for two taste modalities, in accordance with one embodiment of the present invention.

FIG. 2 is a graph representing an exemplary taste recognition profile of an individual for one taste modality. The dashed line represents the taste recognition threshold of the individual.

FIG. 3 is a graph illustrating determination of a taste recognition threshold of a healthy individual for a particular taste modality (salt).

FIG. 4 is of graphs showing salt (a) and sugar (b) taste recognition thresholds of individuals with refractory clinical depression compared with healthy controls. Mann Whitney U test was used to determine statistical significance.

FIG. 5 is of graphs showing perception of salt (a) and sweet (b) intensity as well as pleasantness/unpleasantness of salt (c) and sweet (d) in individuals with refractory depression compared with healthy controls. Intensity and pleasantness/unpleasantness of taste were measured using a visual analogue scale and the units (mm) on the y-axis are millimetres. Mann Whitney U test was used to determine statistical significance.

FIG. 6 is of graphs showing salt (a), sweet (b), sour (c), and bitter (d) taste recognition thresholds of individuals with untreated clinical depression compared with healthy controls. Mann Whitney U test was used to determine statistical significance.

FIG. 7 is of graphs showing salt (a), sweet (b), sour (c), and bitter (d) taste recognition thresholds of 47 individuals with untreated clinical depression before and two hours after an acute SSRI challenge with paroxetine.

FIG. 8 is of graphs showing salt (a), sweet (b), sour (c), and bitter (d) taste recognition thresholds of healthy controls who had not received an acute SSRI challenge and of individuals with untreated clinical depression before and two hours after an acute SSRI challenge with paroxetine. Pre- and post-challenge taste recognition thresholds were compared in the individuals with untreated clinical depression and both values were compared with the taste recognition thresholds of the healthy controls who had not received an acute SSRI challenge. Kruskal Wallis test followed by post-hoc Dunn's tests were used to determine statistical significance, ***p<0.001, ****p<0.0001.

FIG. 9 is of graphs of Receiver Operating Characteristic (ROC) curves for sweet (a), salt (b), bitter (c) and sour (d) taste modalities in individuals with untreated depression.

FIG. 10 is a graph showing the relationship between the sensitivity curve for the sweet taste modality and the specificity curves for the salt, sour and bitter taste modalities as an indicator of response to treatment for depression. The y axis shows the proportion of people who responded to drug treatment that showed a change in taste of either direction in response to the drug challenge.

### DETAILED DESCRIPTION OF THE INVENTION

The “monoamine theory of depression” suggests that diminished circulating monoamine concentrations and hence neurotransmission of NA, dopamine and 5-HT, and/or a reduction in the sensitivity of their receptors represents a biological basis for depression (Hirschfeld, 2000. J Clin Psychiatry 61 [Suppl 6]:4-6). Doctors are currently unable to ascertain whether a patient presenting with depressive symptoms is biologically depressed (and would benefit from the administration of antidepressants) or whether the symptoms are a result of social and/or external factors (which will not be improved by the administration of an antidepressant).

The methods of the present invention are based on the surprising finding that individuals with previously undiagnosed and untreated clinical depression exhibit an acute response to pharmacological challenge when taste recognition profiles are measured, despite a lack of immediate effect on their symptoms. Therefore, by assessing the taste recognition profiles of an individual, the methods of the present invention can be used as a rapid and objective indicator of monoamine levels and/or monoamine receptor sensitivity in the individual. This information can be used to aid the diagnosis of clinical depression in the individual and as an objective indicator of the appropriate form of treatment. A further advantage is that the methods of the present invention require limited specialist or physician time (for example, the methods can be administered by a health care assistant or alternatively, self-administered). In addition, from the point of view of the individual, the methods of the present invention are painless, safe and non-invasive and engage the individual in the diagnostic process and in the monitoring of their own treatment and progress.

### Products for Use in the Methods of the Invention

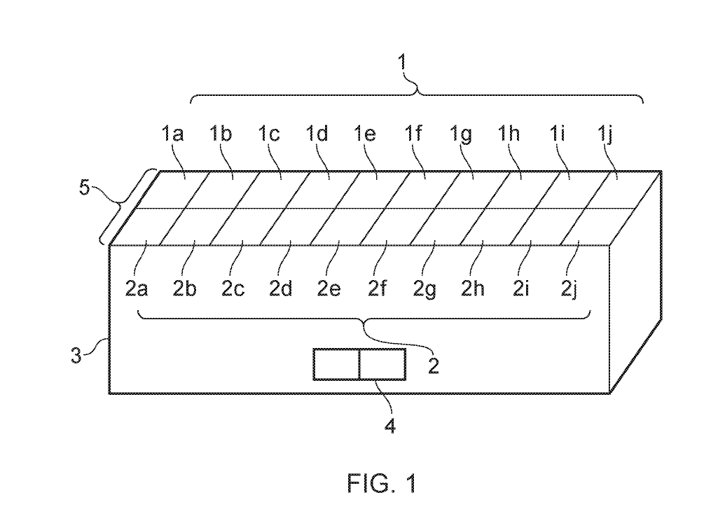

Referring to FIG. 1, a device 3 for determining a taste recognition profile of an individual to a first and second taste modality is shown. The device 3 comprises a plurality of receptacles 5 for holding a plurality of samples 1, 2 in the device 3. The device 3 further comprises an input mechanism 4 for receiving input from the individual. Each sample 1a-1j of the first taste modality has a different concentration of the taste modality in solution, such that a range of concentrations of the first taste modality is provided. Similarly, each sample 2a-2j of the second taste modality has a different concentration of the taste modality in solution, such that a range of concentrations of the second taste modality is provided. In the first embodiment, 10 different concentrations of each taste modality are provided in the device 3. In an alternative embodiment, fewer than 10 concentrations of each taste modality are provided in the device 3 (for example, 5, 6, 7, 8 or 9 concentrations). In a further alternative embodiment, more than 10 concentrations of each taste modality are provided in the device 3 (for example, 11, 12, 13, 14, 15, 20, 25 or 30). Each sample 1a-1j, 2a-2j comprises only one taste modality in solution. In some embodiments, one of the samples of the first and/or second taste modality is water (i.e. represents a blank). The device 3 is configured to enable one sample 1a-1j, 2a-2j to be provided in turn to the individual.

In alternative embodiments, the device 3 is configured such that samples from only a first (i.e. a single) taste modality 1a-1j are provided or such that samples from a third and/or fourth and/or fifth taste modality are provided.

### Methods of the Present Invention

Referring to FIG. 1, use of the device 3 at a first point in time in accordance with a first embodiment of the invention will now be described. The individual to be tested may have symptoms which suggest a psychiatric disorder, such as a clinical anxiety disorder or clinical depression. The method of the first embodiment is performed in two parts and the first part will now be described in relation to a first taste modality.

In use of the device 3, the individual is informed which taste modality is to be tested and a first sample (for example, sample 1h) which has the highest concentration of that taste modality in solution is provided to the individual. By providing the highest concentration first, the individual is able to recognise which taste modality is being tested. In certain situations, the individual is not able to recognise the taste modality in the highest concentration, indicating that the individual has severely reduced taste sensitivity. Such individuals are not suitable for the methods of the present invention. The individual is given a first attempt to recognise the taste modality by applying a proportion of the sample 1h to the individual's protruded tongue. Preferably, the sample 1h is applied to the tip of the individual's protruded tongue. The individual must keep their tongue protruded and indicate whether or not they are able to recognise the taste modality. The results of the first attempt are recorded as a yes/no answer in the input mechanism 4 of the device 3. The individual rinses out their mouth with water to prevent interference between tests. The individual is given a second attempt to recognise the taste modality in sample 1h by repeating the procedure described above with reference to the first attempt. If the individual is confident that they can recognise the taste modality in sample 1h then no further attempts are undertaken. If the individual is not confident, up to five attempts are undertaken. In an alternative embodiment, the individual is confident that they can recognise the taste modality in sample 1h on the first attempt and no second attempt is undertaken.

A second sample of the taste modality (for example, sample 1e) is provided to the individual. The second sample 1e comprises a lower concentration of the taste modality in solution compared to the first sample 1h. The individual is given a first attempt to recognise the taste modality by applying a proportion of the sample 1e to the individual's protruded tongue. Preferably, the sample 1e is applied to the tip of the individual's protruded tongue. The individual must keep their tongue protruded and confirm whether or not they are able to recognise the taste modality. The results of the first attempt are recorded as a yes/no answer in the input mechanism 4 of the device 3. The individual rinses out their mouth with water to prevent interference between tests. The individual is given up to five attempts to recognise the taste modality in the second sample 1e. Each attempt follows the same procedure as described above in relation to the first attempt.

The above procedure as described with reference to the second sample 1e is repeated for a third sample (for example, 1g) of the taste modality. The third sample 1g has a concentration of the taste modality in solution which is different from both the first sample 1h and second sample 1e. In the first embodiment, three samples 1h, 1e, 1g of the taste modality in solution are provided to the individual.

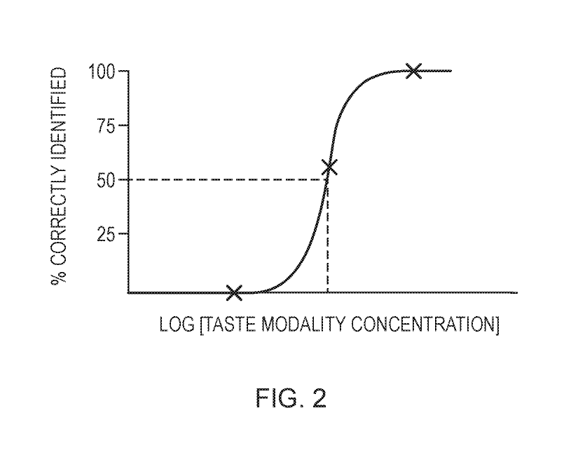

The results from each attempt in respect of each sample 1h, 1e, 1g are recorded as yes/no answers in the input mechanism 4 of the device 3. From this data, the percentage of positive recognition of each concentration of the taste modality is plotted against log solute concentration of the taste modality. Referring to FIG. 2, a curve is then fitted to the data which represents the individual's taste recognition profile to that taste modality. In one embodiment, the curve is an S-shaped curve (see FIG. 2). In the first embodiment, the data from the three samples 1h, 1e, 1g are sufficient to fit the S-shaped curve. A taste recognition threshold of the individual is calculated from the S-shaped curve and recorded. Referring to FIG. 2, the taste recognition threshold is the concentration at which the individual would correctly identify the taste modality 50% of the time (represented by the dashed line in FIG. 2). In an alternative embodiment, the gradient of the S-shaped curve is calculated as part of the taste recognition profile. In one embodiment, the gradient of the S-shaped curve is calculated at the 50% point of the graph. In alternative embodiments, the gradient is calculated at the 25%, 30%, 40%, 60%, 75% point of the graph.

In an alternative embodiment, the data from the three samples 1h, 1e, 1g are insufficient to fit the curve of FIG. 2. In this embodiment, a further 1, 2, 3, 4, 5, 6 or 7 samples of the taste modality in solution are provided to the individual such that a total of 4, 5, 6, 7, 8, 9 or 10 samples are tested. The procedure for each of these further samples is the same as that described above with reference to the second and third samples of the taste modality 1e, 1g. A taste recognition profile is generated from the data as described in the first embodiment and a taste recognition threshold is calculated.

In one embodiment, the first part of the method is complete when there is sufficient data to fit the curve of FIG. 2 for the first taste modality. In this embodiment the first taste modality is sweet. However, in alternative embodiments, the first taste modality is any one of salt, sour, bitter or umami.

In a further embodiment, the above procedure is then repeated for a second taste modality. In this embodiment, the first and second taste modalities are sugar and salt respectively. In alternative embodiments, the second taste modality is any one of sour, bitter or umami. The first part of the method of the invention in respect of the first and second taste modalities is complete when there is sufficient data to fit the curve of FIG. 2 for the two taste modalities.

A pharmaceutical for increasing monoamine neurotransmission is administered to the individual being tested. In the first embodiment, the pharmaceutical for increasing monoamine neurotransmission is paroxetine. Paroxetine is a monoamine reuptake inhibitor, more specifically, a 5-HT-specific reuptake inhibitor (SSRI). In alternative embodiments, the pharmaceutical for increasing monoamine neurotransmission is a different monoamine reuptake inhibitor. In certain embodiments, the pharmaceutical for increasing monoamine neurotransmission is a noradrenaline reuptake inhibitor (NARI). In one embodiment, the noradrenaline reuptake inhibitor is reboxetine.

Two hours after the pharmaceutical challenge, which in this embodiment is the administration of paroxetine, the second part of the method of the invention is undertaken, which is a repeat of the first part of the method of the invention in respect of the first (i.e. sugar) or the first and second taste modalities (i.e. sugar and salt) as described above. A taste recognition profile of the individual prior and subsequent to the pharmaceutical challenge is therefore generated and recorded. In the first embodiment, a taste recognition threshold of the individual prior and subsequent to the pharmaceutical challenge is calculated and recorded.

In the description above, the taste recognition profile is calculated for a taste modality by providing a series of samples of that specific taste modality to the individual. In other words, in embodiments where a taste recognition profile for more than one taste modality is calculated, the taste recognition profile for each taste modality is determined in turn. It is to be understood that the description above is just one example of how a taste recognition profile (including a taste recognition threshold) can be calculated and that other methods are included in the present invention.

In a variant of the embodiment above, a taste recognition profile is calculated for at least one taste modality by providing a series of samples to the individual that comprise a mixed sequence of different taste modalities and concentrations thereof. The individual is asked to identity the particular modality out of a sample of a first, second and optionally a third and/or fourth taste modality that is presented to them. By way of example, in one embodiment, the individual is provided with a sample of a medium/high concentration (thus a perceptible concentration) of a first, second, third and then fourth taste modality and the individual indicates which modality is tasted on each occasion. Subsequently, the individual is provided with samples of a lower concentration of the second, first, fourth and then third taste modality and, again, the individual indicates which modality is tasted on occasion. Subsequent samples of the taste modalities are presented in a similarly mixed sequence and at varying concentrations. In other words, a very low concentration of the third and second taste modality and a higher concentration of the first and fourth taste modality are then presented to the individual. It is to be understood that the sequence described above is by way of example only and that the sequence of the specific taste modalities as well as the concentrations thereof will differ between tests. The concentration of the taste modality provided is influenced by feedback from the results of the previous sample.

The percentage of positive identification of each concentration of a taste modality is plotted against the log solute concentration of the taste modality. The above description of how the taste recognition profile (including the taste recognition threshold) is calculated is relevant to this variant embodiment as well.

Without wishing to be bound by theory, it is thought that by providing a mixed sequence of different taste modalities and concentrations thereof, desensitisation of the individual to a specific taste modality is reduced. Therefore, adopting the procedures of this variant embodiment, can result in lower, and more consistent, taste recognition thresholds being calculated for an individual.

In the first embodiment, the taste recognition threshold of the individual for the first taste modality prior and subsequent to the pharmaceutical challenge is compared and a first change in the taste recognition threshold (“first deltaTRT”) of the individual is calculated as follows.

deltaTRT=taste recognition threshold prior to pharmaceutical challenge−taste recognition threshold subsequent to pharmaceutical challenge

In one embodiment, the deltaTRT of the first taste modality is used to determine monoamine levels and/or monoamine receptor sensitivity in the individual. In one embodiment, the deltaTRT is positive and this represents an increased taste sensitivity to the first taste modality. In other words, there is a decrease in the second taste recognition threshold relative to the first taste recognition threshold. In one embodiment, the percentage change between the second taste recognition threshold relative to the first taste recognition threshold is at least a 10%, 25% or 50% decrease. The increased taste sensitivity is an indication of reduced monoamine levels and/or monoamine receptor sensitivity in the individual prior to the administration of the pharmaceutical for increasing monoamine neurotransmission.

In a further embodiment, the deltaTRT of the first taste modality is compared with a threshold value to determine an increased taste sensitivity. In one embodiment, the positive change in taste recognition threshold is above the threshold value or it is statistically significant compared with the threshold value in order to correspond to “increased taste sensitivity”. In one embodiment, the threshold value is calculated from the deltaTRT values of other individuals suffering from psychiatric, neurological, psychosomatic or physical disorders following an identical taste sensitivity taste to that described above and who were subsequently found to respond positively to treatment with a pharmaceutical for increasing monoamine neurotransmission. In one embodiment, the threshold value is contained within a comparative database.

In an alternative embodiment, the comparative database comprises data from healthy individuals and/or individuals suffering from a psychiatric, neurological, psychosomatic or physical disorder. The data comprise deltaTRT values of individuals following an identical taste sensitivity test to that described above. In one embodiment, the deltaTRT of an individual suffering from a psychiatric, neurological, psychosomatic or physical disorder is significantly different from that of a healthy individual. The data in the comparative database are anonymised.

In one embodiment, an increased taste sensitivity is an indication that the individual is suffering from a psychiatric, neurological, psychosomatic or physical disorder that is suitable for treatment with a pharmaceutical for increasing monoamine neurotransmission.

In an alternative embodiment, the deltaTRT indicates a less than 10% change or a decreased taste sensitivity to the first taste modality. In other words, there is a less than 10% change in a second taste recognition threshold compared with a first taste recognition threshold or an increase in a second taste recognition threshold relative to a first taste recognition threshold, respectively. The less than 10% change or the decreased taste sensitivity to the first taste modality is an indication that the individual is suffering from a psychiatric, neurological, psychosomatic or physical disorder that is not suitable for treatment with a pharmaceutical for increasing monoamine neurotransmission. The aforementioned profile suggests that there is not, or there is not expected to be, an underlying biological cause of the symptoms that could be improved by treatment with a pharmaceutical for increasing monoamine neurotransmission. Therefore, the individual is selected as one who will not be prescribed a pharmaceutical for increasing monoamine neurotransmission. Moreover, it will be noted that the individual is not treated during the test because they are not susceptible to treatment with the pharmaceutical for increasing monoamine neurotransmission. In a preferred embodiment, the individual is instead offered psychological treatment for their symptoms as an alternative form of treatment.

In some embodiments, the first taste modality is the only taste modality tested as described above. In a further embodiment, calculation of a deltaTRT is repeated for the second taste modality. In one embodiment, the deltaTRT of the second taste modality is also used to determine monoamine levels and/or monoamine receptor sensitivity in the individual as described above in relation to the first taste modality. In some embodiments, multiple taste modalities (i.e. two or more) are tested and the deltaTRT of each taste modality is compared against a threshold value to determine monoamine levels and/or monoamine receptor sensitivity in the individual. In one embodiment, the threshold value is different for each different taste modality. In one embodiment, each or the multiple threshold values are contained within a comparative database.

In an alternative embodiment, the first deltaTRT values for both the first and second taste modalities are compared with each other. This comparison is used to determine monoamine levels and/or monoamine receptor sensitivity in the individual. In the first embodiment, a relatively large first deltaTRT in respect of the sweet taste modality (i.e. an increased taste sensitivity) combined with no change or a relatively small first deltaTRT in respect of the salt taste modality (i.e. no change or a less than 10% change in taste sensitivity) is indicative of reduced monoamine levels and/or monoamine receptor sensitivity in the individual. In a preferred embodiment, the relatively large first deltaTRT in respect of the sweet taste modality represents an increase in taste sensitivity of more than 10%. In one embodiment, the first deltaTRT in respect of the sweet taste modality is positive. The relative size of the deltaTRT value in respect of a particular taste modality is determined with respect to the corresponding measurements from other taste modalities in the same individual, which are collected in a comparative database. In other words, the relatively large first deltaTRT represents a reduction in the taste recognition threshold (i.e. an increased taste sensitivity to the sweet taste modality). The relatively small change in the first deltaTRT represents a less than 10% change in the taste recognition threshold (i.e. a less than 10% change in taste sensitivity to the salt taste modality) subsequent to the pharmaceutical challenge relative to the taste recognition threshold prior to the pharmaceutical challenge. In alternative embodiments, a relatively large first deltaTRT in respect of the sweet taste modality combined with a less than 10% change in a first deltaTRT in respect of any one of salt, bitter or sour taste modalities is indicative of reduced monoamine levels and/or monoamine receptor sensitivity in the individual.

In some embodiments, the taste recognition threshold values themselves prior and subsequent to the pharmaceutical challenge are also compared with corresponding measurements from a comparative database to provide further information on the monoamine levels and/or monoamine receptor sensitivity in the individual. The database includes data from healthy individuals and/or from individuals suffering from a psychiatric, neurological, psychosomatic or physical disorder. In this embodiment, the database also comprises the taste recognition threshold values of the individuals following an identical taste sensitivity taste to that described above. In a preferred embodiment, a taste recognition threshold that is higher compared with that of healthy individuals in respect of the first and second modalities prior and/or subsequent to the pharmaceutical challenge is indicative of decreased monoamine levels and/or monoamine receptor sensitivity in the individual. In some embodiments, an appropriate statistical test is used to determine whether or not the taste recognition threshold is significantly higher compared with that of healthy individuals.

In a second embodiment, the first deltaTRT values calculated above for the first and second taste modalities are compared with each other to determine whether the individual suffering from symptoms which suggest a psychiatric disorder, such as a clinical anxiety disorder or clinical depression is not suitable for treatment with a pharmaceutical for increasing monoamine neurotransmission. In this embodiment, a less than 10% change in the first deltaTRT or a negative first deltaTRT (i.e. a less than 10% change or a decreased taste sensitivity) in respect of the sweet taste modality combined with a relatively large change in the first deltaTRT (i.e. an increased or a decreased taste sensitivity) in respect of the salt taste modality indicates that the individual is not suitable for treatment with a pharmaceutical for increasing monoamine neurotransmission. In an alternative embodiment, the relatively large change in the first deltaTRT is in respect of any one of salt, bitter or sour taste modalities. The relative size of the deltaTRT value in respect of a particular taste modality is determined with respect to the corresponding measurements from other taste modalities in the same individual, which are collected in a comparative database. The aforementioned profile suggests that there is not, or there is not expected to be, an underlying biological cause of the symptoms that could be improved by treatment with a pharmaceutical for increasing monoamine neurotransmission. Therefore, the individual is selected as one who will not be prescribed a pharmaceutical for increasing monoamine neurotransmission. Moreover, it will be noted that the individual is not treated during the test because they are not susceptible to treatment with the pharmaceutical for increasing monoamine neurotransmission. In a preferred embodiment, the individual is offered psychological treatment for their symptoms as an alternative form of treatment.

In a third embodiment, an individual tested in accordance with the first embodiment receives treatment for their symptoms in the form of an antidepressant. In order to monitor their progress, the method of the present invention further comprises at a second point in time, the steps of determining a taste recognition profile of the individual for the first or the first and second taste modalities prior and two hours after the pharmaceutical challenge, which in this embodiment is the administration of paroxetine. In this embodiment, taste recognition thresholds are also determined in accordance with the method described above in relation to the first embodiment. The taste recognition profiles are compared to determine a second change in taste recognition profile of the individual to the first or the first and second taste modalities. In this embodiment, the taste recognition thresholds are also compared to determine a second change in taste recognition threshold of the individual to the first and second taste modalities (“a second deltaTRT”). The second deltaTRT values for the first or for both the first and second taste modalities are used or compared with each other in accordance with the methods described above in relation to the first embodiment.

Furthermore, in some embodiments, the first deltaTRT from the first embodiment is compared with the second deltaTRT from this embodiment to determine a difference in the deltaTRT over time for the first taste modality or for each of the first and second taste modalities. In some embodiments, the taste recognition profiles and the taste recognition thresholds calculated prior and subsequent to the pharmaceutical challenge and/or the first and/or second deltaTRT are compared with corresponding measurements from a comparative database to determine monoamine levels and/or monoamine receptor sensitivity in the individual. The database includes data from healthy individuals and/or from individuals suffering from a psychiatric, neurological, psychosomatic or physical disorder. In one embodiment, the database comprises the taste recognition profiles of the individuals following an identical taste sensitivity taste to that described above. In an alternative embodiment, the database comprises first and/or second deltaTRT values of individuals following an identical taste sensitivity taste to that described above. In one embodiment, the comparative database consists of one or more threshold values against which relevant measurements are compared. This comparison is used to determine monoamine levels and/or monoamine receptor sensitivity in the individual. Furthermore, the difference between the first deltaTRT of the first embodiment and the second deltaTRT of this embodiment is also compared with corresponding measurements from a comparative database. The comparisons above provide information which is used to monitor the treatment and progress of the individual. In this embodiment, the database also comprises corresponding differences between the first and second deltaTRTs of individuals following an identical taste sensitivity taste to that described above.

In a fourth embodiment, an individual tested in accordance with the first embodiment receives treatment for their symptoms in the form of an antidepressant. In order to monitor their progress and determine a change in monoamine levels and/or monoamine receptor sensitivity in the individual, the method of the present invention further comprises: at a first point in time, determining a first taste recognition profile of the individual for at least one taste modality after administration of a pharmaceutical for increasing neurotransmission (i.e. the on-going antidepressant treatment), which in this embodiment is the administration of paroxetine; at a second point in time, determining a second taste recognition profile of the individual for the at least one taste modality after administration of a pharmaceutical for increasing neurotransmission (i.e. on-going the antidepressant treatment), which in this embodiment is the administration of paroxetine; and comparing the first taste recognition profile with the second taste recognition profile to determine the change in monoamine levels and/or monoamine receptor sensitivity in the individual. In a manner analogous to that described above in relation to the first embodiment and the third embodiment, the first and second taste recognition profiles of this embodiment and/or the change between them is compared with corresponding measurements from a comparative database to provide further information on the monoamine levels and/or monoamine receptor sensitivity in the individual and/or changes therein. In a variant of the fourth embodiment, the method is performed on an individual receiving treatment for their symptoms in the form of an antidepressant but who has not been tested in accordance with the first embodiment.

The method of the third or fourth embodiment may be repeated at a third, fourth, fifth, sixth, seventh, eighth, ninth, tenth time (there is no upper limit) to provide further information on the treatment and progress of the individual. In one embodiment, the method of the third or fourth embodiment is performed at 8, 15 and/or 29 days after the test in accordance with the first embodiment is performed.

In order to mitigate against the effects of changes in lifestyle that may alter an individual's taste perception over time (for example, smoking), the individual is asked to confirm if any of these changes have occurred when the method of the third or fourth embodiment is performed. These changes in lifestyle are factored into the analysis of the results of the test.

In the embodiments described above, a first taste modality is tested which is sweet or first and second taste modalities are tested, which are sweet and salt taste modalities. In an alternative embodiment, the first or first and second taste modalities are any one or two taste modalities selected from sweet, salt, sour, bitter or umami. In a further alternative embodiment, a third, a fourth and/or a fifth taste modality are tested in addition to a first and second taste modality. These first, second, third, fourth and fifth taste modalities are selected from sweet, salt, sour, bitter, umami. At any point in the embodiments described above, a sample which does not comprise a taste modality (i.e. water) can be provided to the individual. This may be performed if there is a concern that the individual is attempting to manipulate the test.

In one embodiment, the sweet taste modality is sucrose, the bitter taste modality is quinine hydrochloride, the salt taste modality is sodium chloride (NaCl) and/or the sour taste modality is citric acid (C₆H₈O₇). In a preferred embodiment, the range of concentrations of the different taste modalities is as follows: sweet (1M-300 μM sucrose); bitter (300 μM-0.3 μM quinine hydrochloride); salt (1M-0.2 mM NaCl); sour (100 mM-0.56 mM C₆H₈O₇). In an alternative embodiment, the sour taste modality is hydrochloric acid (HCl) and the range of concentrations used is 100 mM-0.56 mM HCl. In one embodiment, the concentration range for each taste modality is independently selected from the above.

In embodiments described above, the second part of the method (i.e. the taste sensitivity test performed after the pharmaceutical challenge) is undertaken at two hours after the pharmaceutical challenge. In an alternative embodiment, the second part of the method is undertaken at 30 minutes to 1 hour or 1, 3, 4 or 5 hours after the pharmaceutical challenge.

It is to be understood that in certain embodiments, administration of a physical sample to determine a taste recognition profile is not necessary as long as the appropriate nerves responsible for the perception of taste are induced.

The principle of the approach described herein is based on monoamine neurotransmitters influencing both taste perception in the taste bud and mood in the higher brain centres. Without wishing to be bound by theory, it is believed that an acute response to the pharmaceutical challenge is exhibited by individuals when taste recognition thresholds are measured despite a lack of immediate effect on their psychiatric, neurological, psychosomatic or physical symptoms because the effect of antidepressants altering plasma concentrations of 5HT and NA affects the taste perception of taste bud cells rather than in higher brain centres initially. Pharmacokinetic study of SSRIs and NA-RIs (serotonin reuptake inhibitors and noradrenaline reuptake inhibitors (i.e. antidepressants)) show that peak plasma concentrations occur at one to two hours (Finley, 1994 Ann Pharmacother 28:1359-1369; Hendershot et al., 2001 Psychopharmacology (Berl) 155:148-153) whereas peak CNS (central nervous system) effects, such as sleep disturbance or motor effects, occur six to eight hours after administration (Saletu et al., 1991 Sleep 14:439-447; Hindmarch, 1997 Eur Neuropsychopharmacol. 7 Suppl 1:S17-21; discussion S71-3; Loubinoux et al., 2002 Neuroimage. 15; 27(2):299-313). This research suggests that at two hours, plasma drug levels are much greater than brain drug levels and peripheral effects (such as in the taste bud cells) are more pronounced than central effects.

It is to be understood that the present invention is not limited to determining monoamine levels and/or monoamine receptor sensitivity in an individual. A large number of neurotransmitters contribute to mental state as well as to taste perception in humans. For example, Noradrenaline (NA) (otherwise known as norepinephrine, NE), Serotonin (5HT) and Opiates (Roper Semin Cell Dev Biol. 2013 24(1): 71-79; Green et al. J Psychopharmacol. 2013 27(3):265-75). Therefore, the present invention is also applicable to determining the levels and/or receptor sensitivity of these neurotransmitters in an individual as well. As such neurotransmitters are involved in a range of psychiatric, neurological, psychosomatic and physical disorders, the present invention can be used to determine whether an individual suffering from one of these disorders is not suitable for treatment with a pharmaceutical for increasing said neurotransmission.

### Examples

### Materials and Methods

1) Protocol for Taste Test

At each visit, individuals with clinical depression and some healthy volunteers completed various questionnaires to determine their mood status, such as Spielberger State and Trait, Beck Depression Inventory, Montgomery-Asberg Depression Rating Scale (MADRS) and Hamilton Rating Scale for Depression (HAM-D).

Taste responses to sweet, salt, bitter and sour stimuli were then determined. Individuals were informed of which taste modality (sweet, bitter, salt, sour) they were receiving but were given no indication of whether the tester thought they would be able to recognise the taste or not.

The taste modalities were applied in solution to the tip of the tongue, using a cotton bud saturated with the solution at room temperature and placed on the tongue for approximately 5 seconds. Without closing their mouth the subject was asked to indicate whether or not they could taste the stimulus at that concentration. Between each stimulus application a 20 second inter-stimulus interval was adhered to during which the subject would rinse their mouth with approximately 25 ml of deionized water.

Each concentration of stimulus was presented to the subject five times in total, and the percentage of positive responses at each concentration determined. Subjects were first presented with a solution above threshold and thereafter, stimuli were presented in a pseudorandom order in concentrations representing ¼ log steps between the lowest (undetectable, 0% detection) to the highest (always detectable, 100% detection) concentrations, with each concentration being presented 5 times.

This protocol was adopted to minimise both adaptation to the stimulus and guessing by the subject.

The range of concentrations for each taste modality was different, as detailed below:
Sweet (1M-300 μM sucrose)
Bitter (300 μM-0.3 μM quinine hydrochloride)
Salt (1M-0.2 mM NaCl)
Sour (100 mM-0.56 mM citric acid; C₆H₈O₇).

The solutions of the taste modalities were prepared shortly prior to testing and kept refrigerated at 4° C. between trials.

Some individuals were also tested for their perception of intensity, and pleasantness of the taste modalities (as described at point 4) below).

2) Protocol for Pharmaceutical Challenge

Psychophysical taste functions were determined before and two hours after administration of a serotonin specific reuptake inhibitor (SSRI; paroxetine 20 mg), or an inactive placebo (lactose, only in some healthy volunteers).

Taste psychometric functions of percentage positive taste recognition against log solute concentration were generated for each individual for each visit to determine individual responses to drug intervention (as described at point 3) below).

Taste psychometric functions before and after paroxetine administration were also determined for all the individuals with previously untreated depression. From these curves, taste recognition thresholds (the concentration at which the subject would recognize the taste 50% of the time) were calculated before and after challenge in order to determine the degree of change in taste recognition that occurred (Heath et al., (2006) Journal of Neuroscience 26(49): 12664-71).

3) Taste Recognition Threshold

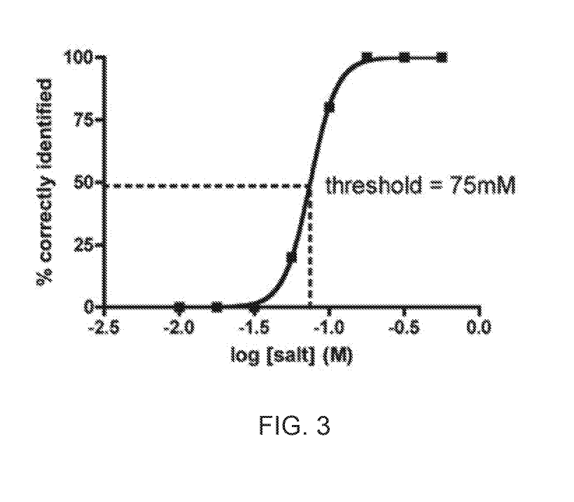

Taste recognition thresholds were determined by testing a solution of a taste modality (sweet, salt, sour, bitter) on the tip of the tongue, and the subject confirmed whether or not they recognised the taste. Referring to FIG. 3, a curve was then constructed of the number of correct identifications against the concentration of the taste in the solution. The taste recognition threshold was calculated from the curve as the concentration at which the subject would correctly identify the taste 50% of the time (Heath, Melichar, Nutt & Donaldson (2006) Journal of Neuroscience 26(49): 12664-71).

4) Intensity and Pleasantness Data

Intensity (strength) and pleasantness/unpleasantness of taste was determined using 1M solutions and the generalised labelled magnitude scale (Bartoshuk et al., (2004) Physiology and Behaviour 83: 109-114) anchored at “barely detectable” and “strongest imaginable sensation” for intensity, and “most pleasant” and “most unpleasant imaginable” for pleasantness. Subjects swilled 5 ml of taste solution around their mouth for 10 seconds and then rated intensity and pleasantness/unpleasantness on the scales.
5) Statistical Analysis

The percentage of positive results for all the individuals were plotted against the log concentrations of the taste solutions before and after pharmaceutical and placebo treatments.

Standard sigmoidal stimulus-response curves of percentage correct taste identification versus log₁₀ taste modality concentration (M) were used to calculate the recognition threshold. Thresholds were compared using parametric or non-parametric ANOVA (>3 groups) following by appropriate post-hoc tests (for example, Bonferroni's tests, Dunn's tests), Mann-Whitney U tests, or t-tests as appropriate.

6) Specificity, Sensitivity, ROC Curves and Likelihood Ratios

Diagnostic tests were evaluated by their sensitivity, and specificity and using ROC curves (Zweig and Campbell (1993) Clinical Chemistry 39: 561-577).

The sensitivity of the challenge test was defined as: the fraction of people in whom the SSRI challenge test resulted in an increased test sensitivity (decreased taste recognition threshold) that then went on to improve with treatment.

The specificity of the challenge test was defined as: the fraction of people in whom the SSRI challenge test resulted in a decreased/unchanged test sensitivity (increased/unchanged taste recognition threshold) that then did not go on to improve with treatment. This is the true negative rate.

In certain situations, two delta TRT values are combined such that the sensitivity of one taste modality test should be high (large fraction responding to the challenge test going on to improvement) and the specificity (otherwise known as the true negative rate) of a second taste modality should be low (low fraction responding to the challenge test not subsequently improving, i.e. not predictive of efficacy).

ROC curves were generated by plotting the true positive rate (sensitivity, the proportions of people showing a change at SSRI challenge who did improve clinically) against the false positive rate (100-specificity, the proportion of people showing a change at SSRI challenge who did not improve clinically). The closer the ROC curve passed to the upper left hand corner of the plot, the greater the overall accuracy of the test (Zweig and Campbell (1993) Clinical Chemistry 39: 561-577).

In other words, the sensitivity of a diagnostic test, in this case the acute SSRI challenge, was the probability that the test result would be positive (a reduction in threshold) when treatment response was positive (improved mood) (true positive rate, expressed as %). On the other hand, the specificity of the diagnostic test was the probability that the test result would be negative when there was no treatment response (true negative rate, expressed as %). There will also be false positives in the clinical group, for example a patient showing a response to acute SSRI challenge who shows no response to treatment, and false negatives, such as a patient showing no response to acute challenge who then shows response to treatment.

The likelihood ratio was the ratio between the probability of a positive test in the presence of positive treatment response and the probability of a positive test in the absence of a treatment response (true positive rate/false positive rate=sensitivity/(100-specificity). A likelihood ratio >1 indicated that the test was associated with the outcome, i.e. a decrease in threshold is associated with a clinical response to treatment.

### Test Subjects

1) Healthy Control Subjects (FIGS. 3 to 6 and 8)

Healthy control subjects (M:F, 20:20, age range 19 to 63 were recruited from the staff and students of a UK Institute of higher education over a number of years (2003 to 2012), for the purposes of determining normal taste perceptual measures. All subjects gave informed consent to protocols approved by the local Research Ethics Committees—REC (Bath and Bristol, UK). Healthy control subjects (n=31) aged between 19 to 56 years were also recruited in Hyderabad, India. Ethical approval for the study was given by the local REC, and all subjects gave informed consent.

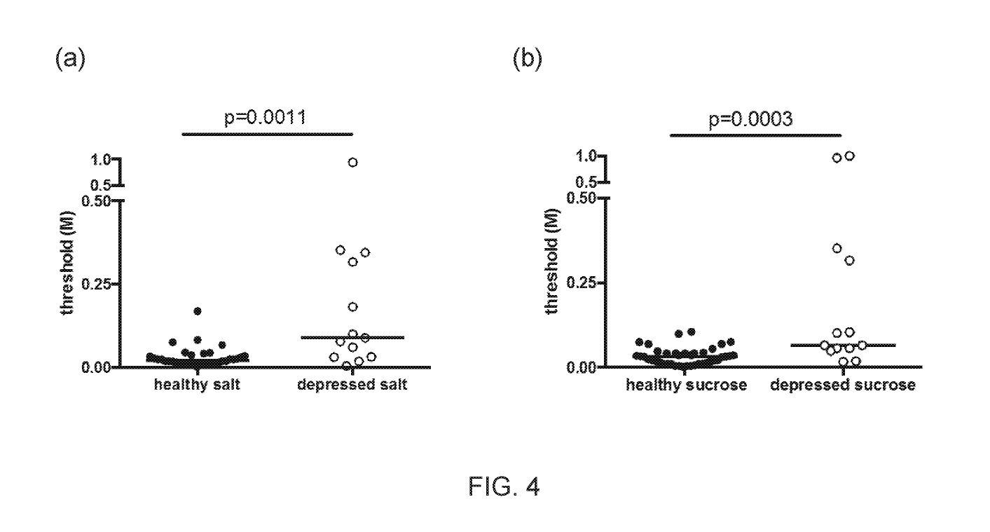

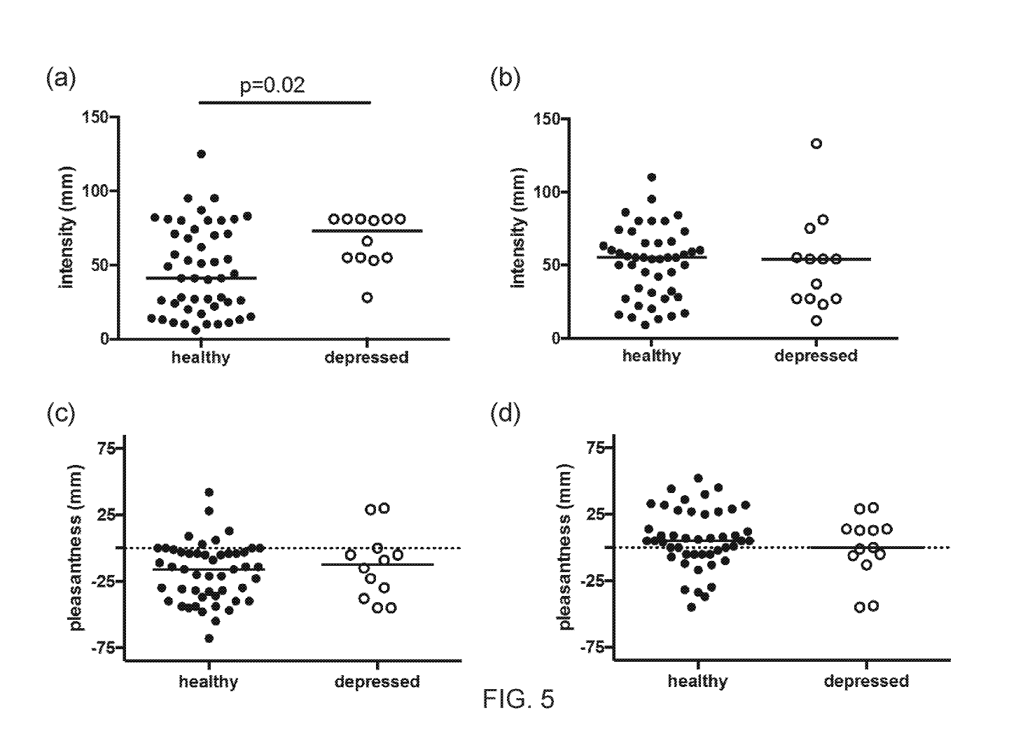

2) Treated Patients with Refractory Depression (FIGS. 4 and 5)

Thirteen patients with long-standing refractory depression (M:F, 6:7) age range 25 to 72 were recruited from a tertiary clinic in Bristol, UK. All subjects gave informed consent, and the study was approved by the local LREC.

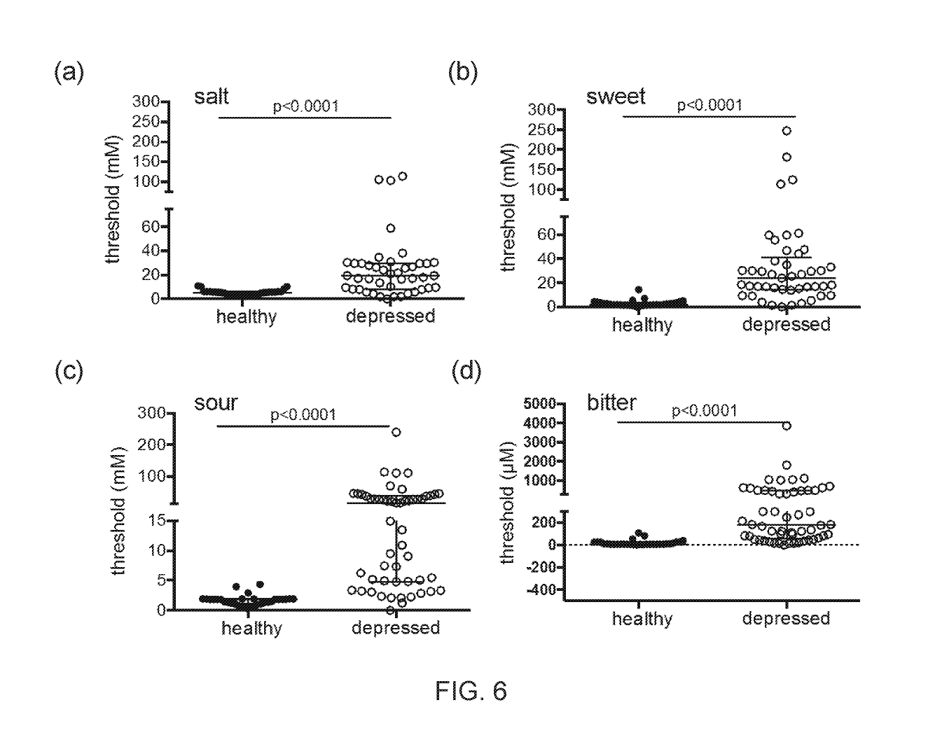

3) Untreated Patients Newly Diagnosed with Clinical Depression (FIGS. 6 to 10; Table 1)

Patients referred to a depression clinic in Hyderabad, India were screened and recruited to the study. They had their taste recognition threshold assessed before and two hours after taking an antidepressant (Paroxetine, SSRI). Follow-up was after 4 weeks.

Exclusion criteria for all studies: abnormal findings of clinical significance on medical or psychiatric history; high caffeine intake (>six cups coffee/day); excessive alcohol intake (>30 units per week); heavy smoking (>20 cigarettes/day); pregnancy; prescribed drugs.

### Example 1: Taste Recognition and Perception in Healthy Controls and Individuals with Refractory Depression

Taste recognition thresholds for salt and sweet taste modalities were calculated for healthy controls and individuals with refractory depression as per the materials and methods. The results are shown graphically in FIG. 4. Referring to FIG. 4, the salt and sweet taste recognition thresholds were significantly higher (i.e. less sensitive) in individuals with refractory depression compared with healthy controls. Statistical significance was determined using Mann Whitney U tests.

Other taste perceptual measures, including intensity and pleasantness/unpleasantness of taste, were measured for salt and sweet taste modalities in healthy controls and individuals with refractory depression as per the materials and methods. The results are shown graphically in FIG. 5. Referring to FIG. 5 (a), salt intensity (i.e. how strong the taste appeared to be) was significantly increased in individuals with refractory depression. In contrast, sweet intensity (FIG. 5 (b)), the unpleasantness of salt (FIG. 5(c)) and the pleasantness of sweet (FIG. 5(d)) were not significantly different from healthy controls. Statistical significance was determined using Mann Whitney U tests.

Therefore, this example demonstrates that individuals with refractory (difficult to treat) depression, who are often on multiple drug therapies, have significantly less sensitivity to both salt and sweet taste modalities (i.e. significantly higher taste recognition thresholds). However, aside from salt intensity, other measures of taste perception (i.e. sweet intensity or pleasantness/unpleasantness of salt or sweet) were not significantly different from healthy controls.

### Example 2: Taste Recognition Thresholds in Healthy Controls and Individuals with Untreated Clinical Depression

Taste recognition thresholds for salt, sweet, sour and bitter taste modalities were calculated for healthy controls and individuals with newly diagnosed and previously untreated clinical depression as per the materials and methods. The results are shown graphically in FIG. 6. Referring to FIG. 6, salt, sweet, sour and bitter taste recognition thresholds were all significantly higher (i.e. less sensitive) in individuals with untreated clinical depression compared with healthy controls. Statistical significance was determined using Mann Whitney U tests.

This example demonstrates that in individuals with newly diagnosed and previously untreated clinical depression, taste recognition thresholds to the four principal taste modalities (salt, sweet, sour and bitter) are significantly higher (i.e. less sensitive).

### Example 3: Taste Recognition Thresholds in Individuals with Untreated Clinical Depression after Acute Antidepressant (SSRI, Paroxetine) Challenge

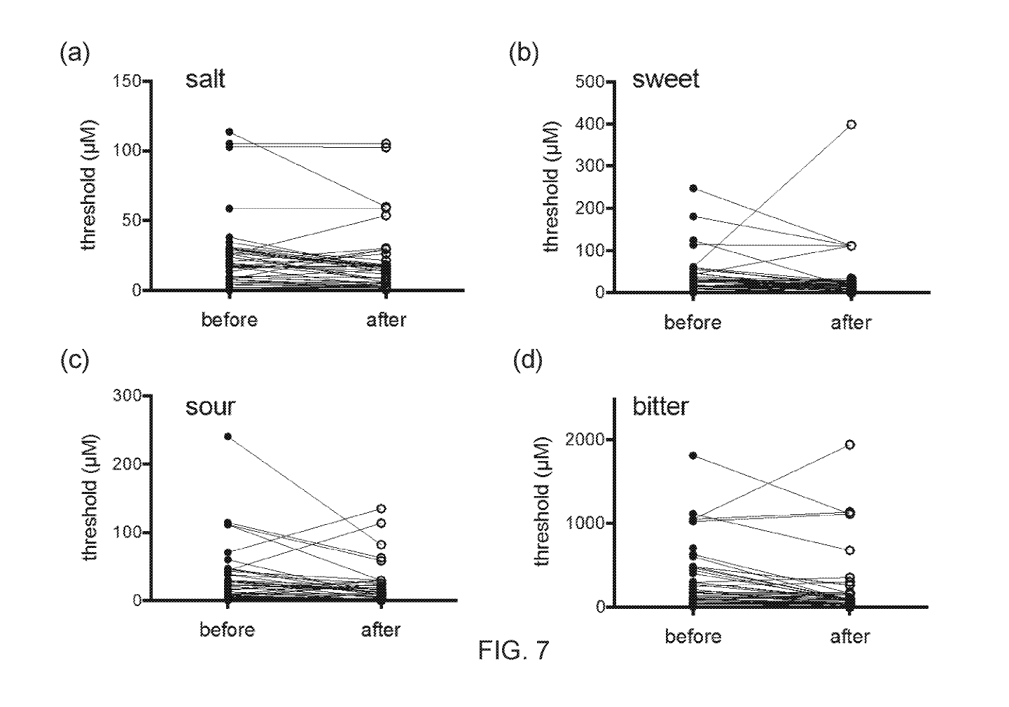

Clinical efficacy of antidepressants against the symptoms of clinical depression may take several weeks to develop. In this example, taste recognition thresholds were calculated in individuals with newly diagnosed and previously untreated clinical depression before and two hours after an acute paroxetine challenge as per the materials and methods. The results are shown graphically in FIG. 7 (n=47, only individuals in whom both measurements were made included; 7 people were excluded as only baseline measurements were made). Referring to FIG. 7, the majority of taste recognition thresholds for each of the salt, sweet, sour and bitter taste modalities were reduced after the acute paroxetine challenge compared with baseline measurements.

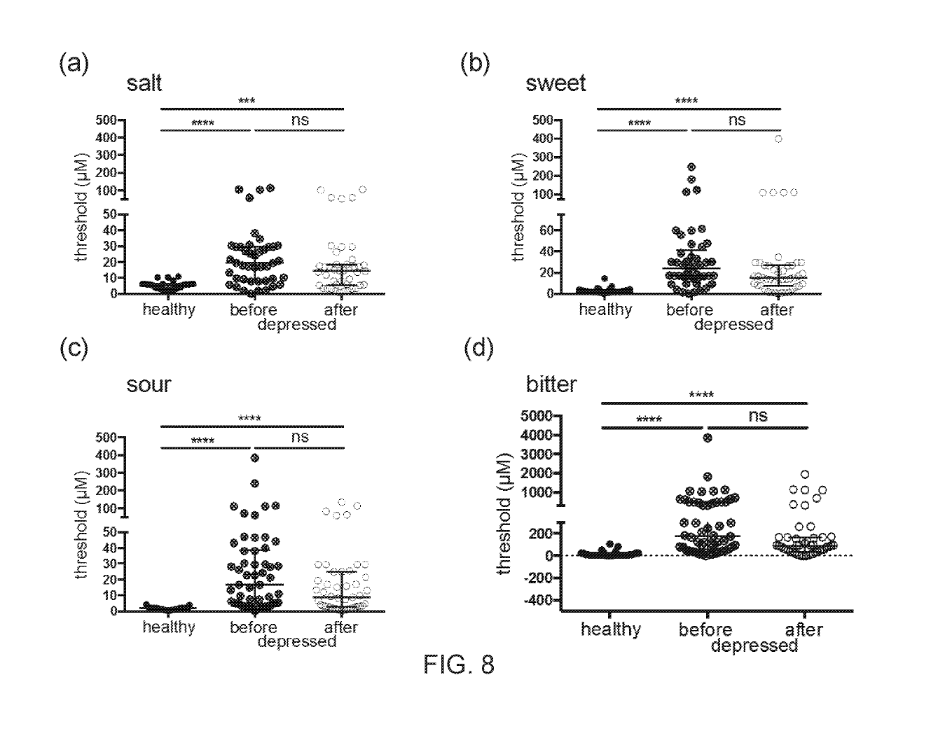

The taste recognition thresholds of individuals with newly diagnosed and previously untreated clinical depression for salt, sweet, sour and bitter taste modalities before and after acute paroxetine challenge were compared with measurements from healthy controls who had not received a paroxetine challenge. The results are shown in FIG. 8. Referring to FIG. 8, after acute paroxetine challenge, although taste recognition thresholds were lowered in people with depression, the post-challenge values were not significantly different from the pre-challenge values (“ns” i.e. not significant). The post-challenge values in the depressed individuals were still significantly higher than those in healthy controls. In fact, for all taste modalities, healthy control values were significantly different from the values from the depressed individuals both before and after paroxetine challenge (***p<0.001, ****p<0.0001). Statistical significance was determined using the Kruskal Wallis test followed by post-hoc Dunn's tests.

This example demonstrates that taste recognition thresholds generally become lower two hours after acute paroxetine challenge in the majority individuals with newly diagnosed and previously untreated depression, although they do not return to the level of those of healthy controls. Therefore, there is an acute response to an antidepressant (in this instance paroxetine) when taste recognition thresholds are measured, despite a lack of immediate effect on depressive symptoms.

### Example 4: Predictive Value of Antidepressant (SSRI, Paroxetine) Challenge in Predicting Therapeutic Efficacy

In patients with untreated clinical depression, taste recognition thresholds and various measures of depression and anxiety (Becks Depression Inventory (BDI) Spielberger State and Trait Inventory, Montgomery-Asberg Depression Rating Scale (MADRS) and Hamilton Rating Scale for Depression (HAM-D)), were determined at baseline, and then treatment was begun at the discretion of the treating clinician. Taste recognition thresholds were also determined after acute paroxetine challenge at the time of first assessment. After four weeks, individuals were then reassessed for their depressive symptoms, and were classified as either a ‘responder’ to treatment (i.e. their depression had begun to decrease) or a ‘non-responder’. A positive response to treatment was defined as an improvement in mood of greater than 10 points on either Becks Depression Inventory (BDI) or the Montgomery-Asberg Depression Rating Scale (MADRS) and an improvement of >5 in the other measure. People without these improvements were classed as ‘non-responders’.

Sensitivity, specificity and likelihood ratios were calculated for each taste modality as per the materials and methods. Sensitivity represents the true positive rate i.e. the fraction of people in whom the SSRI challenge test resulted in an increased taste sensitivity (decreased taste recognition threshold) that then went on to improve with treatment. Specificity indicates the true negative rate i.e. the fraction of people in whom the SSRI challenge test resulted in an increased test sensitivity (decreased taste recognition threshold) that then did not go on to improve with treatment. A likelihood ratio >1 indicates how well the test is associated with the outcome.

TABLE 1
specificity, sensitivity and likelihood ratios for sweet,
salt, sour and bitter taste modalities

| Taste | Sensitivity | Specificity | Likelihood |
|---|---|---|---|
| modality | (%) | (%) | ratio |
| Sweet | 92 | 41 | 1.7 |
| Bitter | 77 | 36 | 1.2 |
| Sour | 77 | 27 | 1 |
| Salt | 46 | 74 | 1.7 |

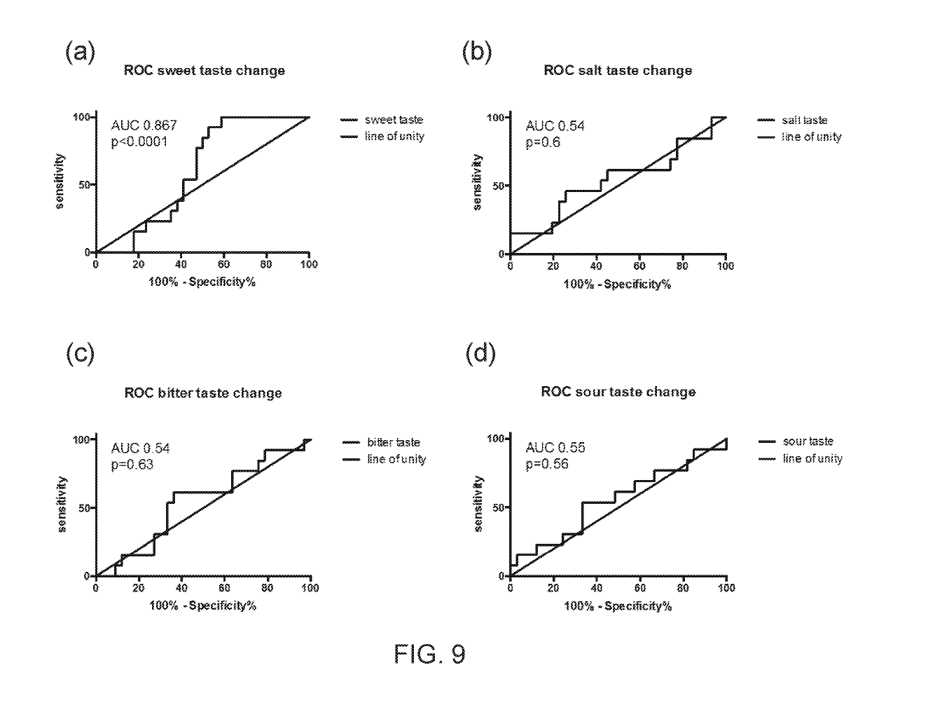

Receiver Operated Characteristic (ROC) curves were generated as per the materials and methods. The results are shown graphically in FIG. 9. ROC for the four taste modalities measured in response to acute paroxetine challenge showed that only the sweet taste modality had a significant leftward shift of the ROC, with an area under the curve (AUC) of 0.867. This indicates that there is an 86.7% probability that people showing the larger reduction in sweet taste recognition thresholds on paroxetine challenge were those who subsequently responded to treatment.

The other three taste modalities (i.e. salt, sour and bitter) did not by themselves show high enough sensitivity to be useful diagnostic tests alone. However likelihood ratios for sweet and salt taste were both high (1.7; as shown in Table 1) and so the outcomes of the responses to both were associated with the response to treatment.

Table 1 demonstrates that, as described above, the magnitude of change in taste recognition threshold (i.e. “deltaTRT”) of the sweet taste modality (before and two hours after paroxetine challenge) had high sensitivity for treatment efficacy (92%), but low specificity (41%). Conversely, the magnitude of change in taste recognition threshold (i.e. “deltaTRT”) of the salt taste modality (before and two hours after paroxetine challenge) had very low sensitivity (46%), too low to be accurate, but had reasonable specificity (74%). The sensitivity and specificity of a diagnostic test can be improved by combining the measurement of more than one parameter.

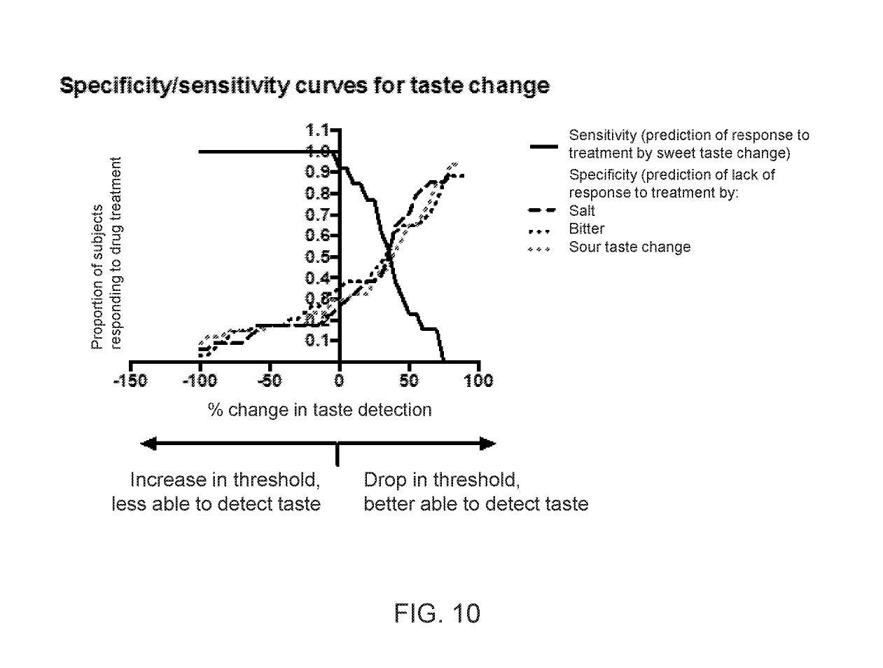

FIG. 10 shows the results of combining the sweet sensitivity curve with salt, bitter and sour specificity curves. Referring to FIG. 10, the sweet sensitivity curve shows the relationship between the sweet deltaTRT (on the x-axis) and the proportion of people who responded to anti-depressant treatment (true positive rate). FIG. 10 demonstrates that 92% of ‘responders’ showed a drop in sweet taste recognition threshold on acute paroxetine challenge (i.e. better able to detect sweet taste).

The specificity curves for salt, sour and bitter taste demonstrate the relationships between the true negative rate (people who showed a change in these taste recognition thresholds and were then identified as ‘non-responders’ to anti-depressant treatment). As the change in the salt, bitter and sour taste recognition thresholds becomes larger (i.e. increased sensitivity) the proportions of ‘non-responders’ incorrectly identified becomes much larger (i.e. the rapid rise of the lines once to the right of 0). A larger proportion of individuals with increased salt threshold failed to respond to treatment (increasing y-axis value to the right of 0). For example, when the increase in sensitivity in salt taste was more than 50%, the proportion of ‘non-responders’ incorrectly identified was above 65%. Thus a smaller salt deltaTRT includes fewer ‘non-responders’ and more ‘responders’. This combined with a high sweet deltaTRT provided a better probability of correct prediction.

Referring to FIG. 10, combining the sweet sensitivity curve with the specificity curves for the other three taste modalities demonstrates the following points. First, the sweet sensitivity curve shows that very few (<10%) of the ‘responders’ had either an increased threshold or no response to acute paroxetine challenge. Those with a drop in sweet taste recognition threshold represent 92% of the ‘responders’ (sensitivity). Second, the specificity of salt, bitter and sour taste changes on acute paroxetine challenge are reasonably equivalent overall, but salt is the most specific (the salt specificity line crosses the y-axis at the lowest value). This demonstrates that a combination of the sweet taste test, with any one of the other taste modalities, but preferably salt, will give adequate sensitivity and reasonable specificity. Approximately 50% of previously untreated people with clinical depression who respond to treatment showed a drop in both sweet and salt taste recognition thresholds, whereas 92% of responders showed a drop in sweet and no change in salt taste recognition threshold. Therefore, the preferred combination for prediction of therapeutic efficacy in terms of specificity and sensitivity, would be a test for sweet and salt deltaTRT, looking for large sweet deltaTRT and small or no salt deltaTRT (certainly <10%).

In conclusion, this example demonstrates that a combination of a change in the sweet taste recognition threshold (a reduction in the taste recognition threshold of any magnitude) and no change in salt threshold (<10% change) represents a predictive test for a positive treatment outcome in individuals with previously untreated clinical depression.

1. A method for determining monoamine levels and/or monoamine receptor sensitivity in an individual, the method comprising the steps of:
(a) determining a taste recognition threshold of the individual for at least one taste modality prior to an administration of a pharmaceutical for increasing monoamine neurotransmission;
(b) determining a taste recognition threshold of the individual for the at least one taste modality subsequent to the administration of the pharmaceutical for increasing monoamine neurotransmission;
(c) performing a comparison between the taste recognition threshold of step (a) and the taste recognition threshold of step (b) to determine a change in taste recognition threshold of the individual to the at least one taste modality; and
(d) performing a comparison between the change in taste recognition threshold and corresponding measurements from a comparative database to determine at least one of monoamine levels and monoamine receptor sensitivity in the individual.

2. The method according to claim 1, wherein the at least one taste modality comprises a sweet taste modality and the comparison between the change in taste recognition threshold and corresponding measurements from the comparative database indicates an increased taste sensitivity for the sweet taste modality, and
wherein the increased taste sensitivity is an indication of at least one of reduced monoamine levels and monoamine receptor sensitivity in the individual prior to the administration of the pharmaceutical for increasing monoamine neurotransmission.

3. The method according to claim 1, wherein the individual is suffering from a psychiatric, neurological, psychosomatic or physical disorder.

4. The method according to claim 2, wherein the individual is suffering from a psychiatric, neurological, psychosomatic or physical disorder, and wherein the increased taste sensitivity for the sweet taste modality is an indication that the individual is suffering from a psychiatric, neurological, psychosomatic or physical disorder that is suitable for treatment with a pharmaceutical for increasing monoamine neurotransmission.

5. The method according to claim 3, wherein the comparison between the change in taste recognition threshold and corresponding measurements from the comparative database indicates a less than 10% change or a decreased taste sensitivity for the at least one taste modality, and
wherein the less than 10% change or the decreased taste sensitivity to the at least one taste modality is an indication that the individual is suffering from a psychiatric, neurological, psychosomatic or physical disorder that is not suitable for treatment with a pharmaceutical for increasing monoamine neurotransmission.

6. The method according to claim 5, wherein the at least one taste modality comprises a sweet taste modality.

7. A method for determining a change in monoamine levels and/or monoamine receptor sensitivity in an individual, the method comprising the steps of:
(a) at a first point in time, determining a first taste recognition threshold of the individual for at least one taste modality subsequent to an administration of a pharmaceutical for increasing monoamine neurotransmission;
(b) at a second point in time, determining a second taste recognition threshold of the individual for the at least one taste modality subsequent to an administration of the pharmaceutical for increasing monoamine neurotransmission; and
(c) comparing the first taste recognition threshold with the second taste recognition threshold to determine the change in at least one of monoamine levels and monoamine receptor sensitivity in the individual.

8. The method according to claim 7, wherein the method further comprises, prior to step (a), the step of:
determining a taste recognition threshold of the individual for at least one taste modality prior to the administration of the pharmaceutical for increasing monoamine neurotransmission; and,
performing a comparison between said taste recognition threshold and at least one of the first taste recognition threshold and the second taste recognition threshold to determine further changes in at least one of monoamine levels and monoamine receptor sensitivity in the individual.

9. A method for determining whether an individual suffering from a psychiatric, neurological, psychosomatic or physical disorder is not suitable for treatment with a pharmaceutical for increasing monoamine neurotransmission, the method comprising the steps of:
(a) determining a taste recognition threshold of the individual for at least one taste modality prior to an administration of the pharmaceutical for increasing monoamine neurotransmission;
(b) determining a taste recognition threshold of the individual for the at least one taste modality subsequent to the administration of the pharmaceutical for increasing monoamine neurotransmission;
(c) performing a comparison between the taste recognition threshold of step (a) and the taste recognition threshold of step (b) to determine a change in taste recognition threshold of the individual to the at least one taste modality; and
(d) performing a comparison between the change in taste recognition threshold and corresponding measurements from a comparative database to determine whether the individual is suffering from a psychiatric, neurological, psychosomatic or physical disorder that is not suitable for treatment with the pharmaceutical for increasing monoamine neurotransmission.

10. The method according to claim 9, wherein the at least one taste modality comprises a sweet taste modality and the comparison between the change in taste recognition threshold and corresponding measurements from the comparative database indicates a less than 10% change or a decreased taste sensitivity for the sweet taste modality,
wherein the less than 10% change or the decreased taste sensitivity to the sweet taste modality is an indication that the individual is suffering from a psychiatric, neurological, psychosomatic or physical disorder that is not suitable for treatment with the pharmaceutical for increasing monoamine neurotransmission.

11. A method for determining whether an individual suffering from a psychiatric, neurological, psychosomatic or physical disorder is not suitable for treatment with a pharmaceutical for increasing monoamine neurotransmission, the method comprising the steps of:
(a) determining a taste recognition threshold of the individual for first and second taste modalities prior to an administration of the pharmaceutical for increasing monoamine neurotransmission;
(b) determining a taste recognition threshold of the individual for first and second taste modalities subsequent to the administration of the pharmaceutical for increasing monoamine neurotransmission;
(c) performing a comparison between the taste recognition threshold of step (a) and the taste recognition threshold of step (b) to determine a first change in taste recognition threshold of the individual to the first taste modality and a second change in taste recognition threshold of the individual to the second taste modality; and
(d) performing a comparison between the first change in taste recognition threshold and the second change in taste recognition threshold to determine whether the individual is suffering from a psychiatric, neurological, psychosomatic or physical disorder that is not suitable for treatment with the pharmaceutical for increasing monoamine neurotransmission.

12. The method according to claim 11, wherein the first and second taste modalities are sweet and salt respectively and the comparison of step (d) indicates a relatively small or negative first change in taste recognition threshold and a relatively large second change in taste recognition threshold,
wherein the relatively small or negative first change in taste recognition threshold and the relatively large second change in taste recognition threshold is an indication that the individual is suffering from a psychiatric, neurological, psychosomatic or physical disorder that is not suitable for treatment with the pharmaceutical for increasing monoamine neurotransmission.

13. A sample of a taste modality for use in a method of in vivo diagnosis of a disorder in which monoamine levels and/or monoamine receptor sensitivity is deregulated in an individual, wherein the method comprises the steps of:
(a) contacting the taste modality with the individual's tongue and determining a taste recognition threshold of the individual for the taste modality prior to an administration of a pharmaceutical for increasing monoamine neurotransmission;
(b) contacting the taste modality with the individual's tongue and determining a taste recognition threshold of the individual for the taste modality subsequent to the administration of the pharmaceutical for increasing monoamine neurotransmission;
(c) performing a comparison between the taste recognition threshold of step (a) and the taste recognition threshold of step (b) to determine a change in taste recognition threshold of the individual to the taste modality; and
(d) performing a comparison between the change in taste recognition threshold and corresponding measurements from a comparative database to diagnose a disorder in which at least one of monoamine levels and monoamine receptor sensitivity is deregulated in the individual.

14. The sample for use in the method according to claim 13, wherein the taste modality is a sweet taste modality and the comparison between the change in taste recognition threshold and corresponding measurements from the comparative database indicates an increased taste sensitivity to the sweet taste modality, and
wherein the increased taste sensitivity is an indication of a disorder in which at least one of monoamine levels and/or monoamine receptor sensitivity is deregulated in the individual.

15. The sample for use in the method according to claim 13, wherein the disorder in which at least one of monoamine levels and monoamine receptor sensitivity is deregulated is a psychiatric, neurological, psychosomatic or physical disorder.

16. The method according to claim 3, wherein the psychiatric disorder is clinical depression or a clinical anxiety disorder.

17. The method according to claim 1, wherein the comparison is performed using a means for comparison.

18. The method according to claim 1, wherein the pharmaceutical for increasing monoamine neurotransmission is at least one of dopamine, noradrenaline and serotonin reuptake inhibitor.

19. A method for determining monoamine levels and/or monoamine receptor sensitivity in an individual suffering from a psychiatric, neurological, psychosomatic or physical disorder, the method comprising the steps of:
(a) determining a taste recognition threshold of the individual for at least one taste modality prior to an administration of a pharmaceutical for increasing monoamine neurotransmission;
(b) determining a taste recognition threshold of the individual for the at least one taste modality subsequent to the administration of the pharmaceutical for increasing monoamine neurotransmission; and
(c) performing a comparison between the taste recognition threshold of step (a) and the taste recognition threshold of step (b) to determine a change in taste recognition threshold of the individual to the at least one taste modality,
wherein the change in the taste recognition threshold is used to determine at least one of monoamine levels and monoamine receptor sensitivity in the individual.

20. The method according to claim 19, wherein the change in taste recognition threshold is an increased taste sensitivity to the at least one taste modality and the increased taste sensitivity is an indication of at least one of reduced monoamine levels and monoamine receptor sensitivity in the individual prior to the administration of the pharmaceutical for increasing monoamine neurotransmission.

21. The method according to claim 20, wherein the increased taste sensitivity is an indication that the individual is suffering from a psychiatric, neurological, psychosomatic or physical disorder that is suitable for treatment with a pharmaceutical for increasing monoamine neurotransmission.

22. The method according to claim 19, wherein the change in taste recognition threshold is a less than 10% change or a decreased taste sensitivity to the at least one taste modality, and
wherein the less than 10% change or the decreased taste sensitivity to the at least one taste modality is an indication that the individual is suffering from a psychiatric, neurological, psychosomatic or physical disorder that is not suitable for treatment with a pharmaceutical for increasing monoamine neurotransmission.

23. The method according to claim 19, wherein the at least one taste modality comprises a sweet taste modality.
